(19) United States
(12) Patent Application Publication
(10) Pub. No.: US 2022/0145308 A1
(43) Pub. Date: May 12, 2022

(54) MATERIALS AND METHODS FOR REDUCING NUCLEIC ACID DEGRADATION IN BACTERIA

(71) Applicant: University of Florida Research Foundation, Inc., Gainesville, FL (US)

(72) Inventors: Valerie Anne De Crecy, Gainesville, FL (US); Geoffrey Jean Hutinet, Gainesville, FL (US)

(21) Appl. No.: 17/433,631

(22) PCT Filed: Mar. 10, 2020
(86) PCT No.: PCT/US20/21886

§ 371 Date: Aug. 25, 2021

Related U.S. Application Data
(60) Provisional application No. 62/816,615, filed on Mar. 11, 2019.

(51) Int. Cl. C12N 15/70; C12N 9/10; C12N 9/00; C12N 7/00

(52) U.S. Cl. CPC C12N 15/70; C12N 9/1051; C12N 2795/00051; C12Y 603/0402; C12N 7/00; C12N 9/93

(57) ABSTRACT

The present disclosure is directed to materials and methods for reducing heterologous DNA damage in bacteria (i.e., induce resistance to host restriction enzymes) by modifying the heterologous DNA to include one or more deazapurine bases.

### CROSS-REFERENCE TO RELATED APPLICATIONS

The present application claims the benefit of priority to U.S. Provisional Application No. 62/816,815, filed Mar. 11, 2019, the disclosure of which is incorporated by reference in its entirety.

### STATEMENT OF GOVERNMENT SUPPORT

This invention was made with government support under GM070641 awarded by The National Institutes of Health. The government has certain rights in the invention.

### FIELD OF THE INVENTION

The present disclosure is directed to materials and methods for reducing heterologous DNA damage in bacteria by modifying the heterologous DNA to include one or more deazapurine bases.

### BACKGROUND

DNA that is recognized as foreign to a given cell may be targeted for degradation within the cell, either by its lack of a host-like methylation pattern or by the presence of unusual base modifications relative to the host DNA (Bair and Black, 2007, J Mol Biol 366: 768-778). The subsequent degradation by restriction endonucleases reportedly constitutes effective barriers to the introduction of DNA into bacteria (Briggs et al. Appl. Environ. Microbiol. 1994, 60, 2006-2010; Accetto et al. FEMS Microbiol. Lett. 2005, 247, 177-183; Bair and Black, J. Mol. Biol. 2007, 366, 768-778; Corvaglia et al. Proc. Natl. Acad. Sci. U.S.A. 2010, 107, 11954-11958; Monk et al., 2012, mBio 3(2): e00277-11.doi: 10.1 128/mBio.00277-11).

These endonuclease-based systems are grouped into four main types, type I to type IV, by a number of criteria (Roberts et al. Nucleic Acids Res. 2003, 31, 1805-1812). Systems of type I to type III encompass paired methyltransferase and endonuclease activities, degrading foreign DNA that lacks the proper methylation pattern, whereas the type IV enzymes are endonucleases that only cleave DNA substrates that have been modified (Tock and Dryden, Curr. Opin. Microbiol. 2005, 8, 466-472).

Bacterial transformants provide a key platform for a variety of industrially relevant processes, such as metabolic engineering and biochemical production. However, the introduction and expression of foreign DNA into some bacterial hosts can be an inefficient process. There is a need in the art for new strategies for maximizing the functionality of heterologous DNA in bacteria.

Bacteriophages (phages) are viruses that specifically infect and lyse bacteria. Phage therapy, a method of using whole phage viruses for the treatment of bacterial infectious diseases, was introduced in the 1920s by Felix d'Herelle. Initially, phage therapy was vigorously investigated and numerous studies were undertaken to assess the potential of phage therapy for the treatment of bacterial infection in humans and animals.

With the development of antibiotics in the 1940s, however, interest in phage-based therapeutics declined in the Western world. One of the most important factors that contributed to this decline was the lack of standardized testing protocols and methods of production. The failure to develop industry wide standards for the testing of phage therapies interfered with the documentation of study results, leading to a perceived lack of efficacy as well as problems of credibility regarding the value of phage therapy.

With the rise of antibiotic resistant strains of many bacteria, however, interest in phage-based therapeutics has returned. Even though novel classes of antibiotics may be developed, the prospect that bacteria will eventually develop resistance to the new drugs has intensified the search for non-chemotherapeutic means for controlling, preventing, and treating bacterial infections.

### SUMMARY

In one aspect, described herein is a bacterial cell comprising a heterologous nucleic acid sequence comprising one or more deazapurine bases. In some embodiments, the one or more deazapurine bases are deazaguanine bases (e.g., 7-deazaguanine bases). Exemplary 7-deazaguanine bases include, but are not limited to, 7-amido-7-deazaguanine (ADG), 7-formamidino-7-deazaguanosine (G⁺), 7-cyano-7-deazaguanine (PreQ₀) and 7-aminomethyl-7-deazaguanine (PreQ₁).

In another aspect, described herein is a method of protecting a heterologous nucleic acid sequence from cleavage by restriction enzymes in a host bacterium, the method comprising modifying the heterologous nucleic acid sequence to incorporate one or more deazaguanine bases; and introducing the modified heterologous nucleic acid sequence into the host bacterium, thereby protecting the heterologous nucleic acid sequence from cleavage by restriction enzymes in the host bacterium. In some embodiments, the modifying step occurs in vitro. In this regard, in some embodiments, the modifying step comprises mixing the heterologous nucleic acid sequence with at least one enzyme that is involved in introducing deazaguanine bases in DNA for a time sufficient to promote modification of the heterologous nucleic acid sequence.

In some embodiments, the modifying step comprises introducing the heterologous nucleic acid into a bacterial cell that has been modified to encode at least one enzyme that is involved in introducing deazaguanine bases in DNA.

Exemplary enzymes that are involved in introducing deazaguanine bases in DNA include, but are not limited to, DpdA and Gat-QueC encoded by Enterobacteria phage 9g.

### BRIEF DESCRIPTION OF THE FIGURES

FIG. 1: Queuosine and Archeosine synthesis pathways. PreQ₀ is synthesized from GTP in both bacteria and archaea through FolE, QueD, QueE and QueC as shown. In most bacteria, four more enzymatic steps lead to the insertion of Q in tRNAs at position 34 (dashed square on lower left). In archaea, PreQ₀ is transferred to position 15 of tRNA before being modified to G⁺ (dashed rectangle on lower right). Bases identified in this study that are found in phage DNA include PreQ₁, PreQ₀, ADG and G⁺. Molecule abbreviations: guanosine tri-phosphate (GTP), dihydroneopterin triphosphate (H₂NTP), 6-carboxy-5,6,7,8-tetrahydropterin (CPH₄), 5-carboxy-deazaguanine (CDG), 7-amido-7-deazaguanine (ADG), 7-cyano-7-deazaguanine (PreQ₀), 7-aminomethyl-7-deazaguanine (PreQ₁), queuosine (Q) and archaeaosine (G⁺).

FIGS. 2A-2C. FIG. 2A is a Northern blot of an acrylamide electromobility gel shift assay showing the tRNA-Q complementation of E. coli mutants by Enterobacteria phage 9g orthologs. The WT strain modifies the tRNA_{Asp} with Q and is shifted in its migration (Q line), but the E. coli mutant strains (ΔfolE, ΔqueD, ΔqueE, ΔqueC and Δtgt) are not modified and migrate further (no Q line). In each mutant, the Enterobacteria phage 9g orthologs has been expressed in trans. The complementation of Δtgt by E. coli tgt is shown as positive control of complementation. FIG. 2B is an agarose gel of EcoRI digestion of plasmid extracted from different strains of E. coli (WT, ΔqueC, ΔqueD, Δtgt) expressing variant of pBAD33 and pBAD24 (empty plasmid, 0, encoding Enterobacteria phage 9g dpdA, A, or encoding Enterobacteria phage 9g gat-queC, C). EcoRI cut pBAD24 once (4542 bp fragment) and pBAD33 twice (2479 bp and 2873 bp fragments). The resulting sizes for the digestion of pBAD24 are 5971 bp and 5509 bp when qat-queC or dpdA is inserted, respectively. For pBAD33, the 2873 bp fragment stays unchanged but the 2479 bp fragment shifts to 3911 when gat-queC is inserted and 3449 bp when it is dpdA. The presence (+) or absence (−) of the modifications identified (dPreQ₀ and dG⁺) by mass spectrometry are indicated under the gel. FIG. 2C is an agarose gel of uncut (0) or EcoRI cut (D) pGH39/pGH66 couple of plasmids extracted from a WT strain of E. coli repressed in 0.4% glucose (Glu) or induced in 0.4% arabinose (Ara).

FIG. 3. Genomic context of the dpdA and dG+/PreQ0 biosynthesis pathway genes of Enterobacteria phage 9g, Streptococcus phage Dp-1, Vibrio phage nt-1, Mycobacterium phage Rosebush, Escherichia phage CAjan, Salmonella phage 7-11, Mycobacterium phage Orion and Halovirus HVTV-1. The genes are colored by functions: white is DpdA, shades of grey are the biosynthetic pathway of PreQ₀, and the genes coding for aminotransferases that synthetize G⁺ from PreQ₀. In black are all other proteins. (*) Note that Streptococcus phage Dp-1 is grouped in the dG+ biosynthesis pathway in the bioinformatics analysis but it does not produce this modification.

FIGS. 4A-4C are gels showing the restriction pattern with different restriction enzymes on the DNA of Enterobacteria phage 9g (FIG. 4A), Mycobacterium phage Rosebush (FIG. 4B) and Enterobacteria phage CAjan (FIG. 4C), as well as the representation of the expected restriction pattern.

FIG. 5 provides a proposed synthesis pathway of the 2′-deoxy-7-deazaguanine modification. Percentages of modification identified for each phage are shown in boxes next to the modification of interest. Molecule abbreviations: guanosine tri-phosphate (GTP), 7-cyano-7-deazaguanine (PreQ₀), 2′-deoxy-7-cyano-7-deazaguanosine (dPreQ₀), guanine (G), 2′-deoxyguaonosine (dG), 2′-deoxy-7-aminomethyl-7-deazaguanosine (dPreQ₁), 2′deoxy-7-amido-7-deazaguanosine (dADG) and 2′-deoxyarchaeaosine (dG⁺).

FIGS. 6A-6C are schematics showing means of introducing the modifications described herein. (A) The modified mobile genetic elements (MGE) will resist the degradation system from the bacteria of interest compared to the unmodified MGE, and then further be replicated and modified by the natural modification system of the bacteria. (B) In vivo modification strategy: an unmodified MGE is introduced in the strain expressing Enterobacteria phage 9g dpdA and gat-queC. The resulting modified MGE is then extracted. (C) As an in vitro modification strategy, an unmodified MGE DNA is mixed with the purified Enterobacteria phage 9g DpdA and Gat-QueC protein and PreQ₀. The resulting modified MGE is then purified.

### DETAILED DESCRIPTION

The present disclosure is based, at least in part, on the discovery that a deoxyribonucleic acid (DNA) sequence comprising one or more 7-deazaguanine modifications dramatically decreases the susceptibility of the DNA to endonucleases in bacterial host restriction-modification systems (RM) compared to the same nucleic acid sequence without the 7-deazaguanine modifications. Restriction-modification systems are one of the major defense systems for bacteria to prevent the invasion by foreign nucleic acids⁵, such as phages, plasmids or integrons. Modifying nucleic acids (e.g., DNA) to incorporate the 7-deazaguanine modifications disclosed herein results in increased functionality or productivity of bacterial transformants because the modified DNA is less susceptible to host bacterial endonucleases.

Wild type bacteria encode for multiple defense systems against mobile genetic elements (MGEs). Many of these MGEs are used as tools for genetic engineering applications or as weapons against pathogens. Hence, the availability of a method that would protect these MGEs from bacterial defenses, particularly restriction enzymes, would greatly enhance their effectiveness. As demonstrated herein, nucleic acids (e.g., DNA) modified by dPreQ₀, dPreQ₁ or dG⁺ are protected from cleavage by a wide variety of restriction enzymes.

In one aspect, described herein is a bacterial cell (or bacterium) comprising a heterologous nucleic acid sequence comprising one or more deazaguanine bases. In some embodiments, the deazaguanine bases are 7-deazaguanine bases. Exemplary 7-deazaguanine bases include, but are not limited to, 7-amido-7-deazaguanine (ADG), 7-cyano-7-deazaguanine (PreQ₀), 7-formamidino-7-deazaguanosine (G⁺) and 7-aminomethyl-7-deazaguanine (PreQ₁).

In some embodiments, modifying the heterologous nucleic acid with one or more deazaguanine bases results in resistance to degradation by one or more restriction enzymes. In some embodiments, the one or more restriction enzymes is EcoRI (E. coli), EcoRII (E. coli), BamHI (B. amyloiquefaciens), HindIII (H. influenzae), NotI (N. otitidis), HinFI (H. influenzae), Sau3AI (S. aureus), PvuII (P. vulgaris), SmaI (S. marcescens), HaeIII (H. aegyptius), HgaI (H. gallinarum), AliI (A. luteus), EcoRV (E. coli), EcoP15I (E. coli), KpnI (K. pneumonia), PstI (P. stuartii), SacI (S. achromogenes), SalI (S. albus), Seal (S. caespitosus), SpeI (S. natans), SphI (S. phaeochromogenes), StuI (S. tubercidicus) and/or XbaI (X. badrii). Optionally, the heterologous nucleic acid comprising one or more deazaguanine bases is resistant to degradation by one or more of EcoRI, EcoRII, EcoRV and EcoP15I when transformed in E. coli.

The term “heterologous nucleic acid” is a nucleic acid that is not normally present in a particular wild type host cell. The bacterium has been “genetically modified” or “transformed” or “transfected” by heterologous nucleic acid when such nucleic acid(s) has been introduced inside the cell. Nucleic acids include DNA and RNA; can be single- or double-stranded; can be linear, branched or circular; and can be of any length. The heterologous nucleic acid described herein can be any DNA of interest. The DNA may be of genomic, cDNA, semisynthetic, synthetic origin, or any combinations thereof. The heterologous nucleic acid may encode any polypeptide having biological activity of interest or may be a DNA involved in the expression of the polypeptide having biological activity, e.g., a promoter. The heterologous nucleic acid encoding a polypeptide of interest may be obtained from any prokaryotic, eukaryotic, or other source. For purposes of the present disclosure, the term “obtained from” as used herein in connection with a given source shall mean that the polypeptide is produced by the source or by a cell in which a gene from the source has been inserted.

In some embodiments, the heterologous nucleic acid is a mobile genetic element. The term “mobile genetic element” or “MGE” as used herein refers to genetic elements that are not bound to a bacterial host and have the ability to move from one bacterial host to another. In some embodiments, the movement of DNA is within genomes (intracellular mobility). In some embodiments, the movement of DNA is between cells (intercellular mobility). Examples of MGEs include, but are not limited to, transposons, plasmids, bacteriophage nucleic acids, and pathogenicity islands. The MGE can be naturally occurring or engineered. The MGE can be cell-type specific, tissue specific, organism specific, or species specific (e.g., bacteria specific or human specific). The MGE can also be non-specific with respect to cell-type, tissue, organism and/or species.

A nucleic acid may be modified to incorporate one or more deazapurine bases in a cell-free environment or may be similarly modified in a bacterial cell. In some embodiments, the nucleic acid is modified in a bacterial cell. For example, in some embodiments, a nucleic acid (e.g., MGE) is introduced into a bacterial cell (e.g., E. coli, B. cereus, or B. subtilis) that has been modified to encode a transglycosidase (e.g., dpdA gene) and an amidotransferase (e.g., gat-queC gene) from Enterobacteria phage 9g and express their respective proteins, DpdA and Gat-QueC. The bacterial cell in its native state expresses additional enzymes (e.g., FolE, QueD, QueE and QueC) that are involved in the four first steps of PreQ₀ synthesis. The expression of these native enzymes with a transglycosidase (and an amidotransferase) results in guanine(s) in the nucleic acid (e.g., MGE) being replaced with 7-cyano-7-deazaguanine (PreQ₀) and 7-formamidino-7-deazaguanosine (0). The modified nucleic acid (comprising one or more deazapurine bases) can be collected by lysing the bacterial cell, and then subsequently introduced into a strain of interest.

In some embodiments, the nucleic acid is modified in a cell free environment. In this regard, isolated and purified transglycosidase (e.g., DpdA) and amidotransferases (e.g., Gat-QueC) are mixed with the nucleic acid (e.g., MGE) and the PreQ₀ base (commercially available) for a time and temperature sufficient to promote modification of the nucleic acid by 7-formamidino-7-deazaguanosine (G⁺). The modified nucleic acid (comprising one or more deazapurine bases) can then be purified and introduced into a strain of interest. The use of DpdA alone will provide a nucleic acid modified with dPreQ₀.

In some embodiments, a dGPT in a nucleic acid is modified into include a 7-substituted dazapurine dGTP, which DNA polymerases can use as a dNTP substrate to be integrated into newly created DNA (e.g., by PCR) (Cahove et al., ACS Chem. Biol. 11:3165-3171, 2016, the disclosure of which is incorporated herein by reference in its entirety).

In some embodiments, the heterologous nucleic acid is incorporated into a plasmid or other suitable expression vector (e.g., a bacteriophage-based vector). As used herein, the term “plasmid” or “vector” refers to an extrachromosomal nucleic acid, e.g., DNA, construct that is not integrated into a bacterial cell's chromosome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a modified heterologous nucleic sequence e.g., a nucleotide sequence comprising one or more 7-deazaguanine bases.

The vector may contain one or more (e.g., two, several) selectable markers that permit easy selection of transformed bacterium (or bacterial cell). A selectable marker is a gene the product of which provides for biocide or viral resistance, resistance to heavy metals, prototrophy to auxotrophs, and the like. Examples of selectable markers include, but are not limited to, the dal genes from Bacillus subtilis or Bacillus licheniformis, or markers that confer antibiotic resistance such as ampicillin, chloramphenicol, kanamycin, or tetracycline resistance. Suitable markers for yeast host cells are ADE2, HIS3, LEU2, LYS2, MET3, TRP1, and URA3.

General methods, reagents and tools for transforming (e.g., bacteria) can be found, for example, in Sambrook et al (2001) Molecular Cloning: A Laboratory, Manual, 3rd ed., Cold Spring Harbor Laboratory Press, New York. Methods, reagents and tools for transforming yeast are described in “Guide to Yeast Genetics and Molecular Biology,” C. Guthrie and G. Fink, Eds., Methods in Enzymology 350 (Academic Press, San Diego, 2002).

In some embodiments, introduction of the modified heterologous nucleic acid sequence (or vector comprising the modified heterologous nucleic acid sequence) of the present disclosure into a host cell is accomplished by calcium phosphate transfection, DEAE-dextran mediated transfection, electroporation, or other common techniques (See Davis et al., 1986, Basic Methods in Molecular Biology, which is incorporated herein by reference). In one embodiment, a preferred method used to transform E. coli strains is electroporation and reference is made to Dower et al., 1988) NAR 16: 6127-6145. Indeed, any suitable method for transforming host cells can be used. It is not intended that the present disclosure be limited to any particular method for introducing the modified heterologous nucleic acids into host cells.

In some embodiments, the bacterial cell (or bacterium) is modified via CRISPR (Clustered Regularly Interspaced Short Palindromic Repeats) technology to express the modified heterologous nucleic acid. A CRISPR genomic locus can be found in the genomes of many bacteria and archaea. The CRISPR locus encodes products that function as a type of immune system to help defend the cell against foreign invaders, such as virus and phage. There are three stages of CRISPR locus function: integration of new sequences into the locus, biogenesis of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” The repeats can form hairpin structures and/or comprise unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA comprises a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also comprises polynucleotide sequences encoding CRISPR Associated (Cas) genes. Cas genes encode endonucleases involved in the biogenesis and the interference stages of crRNA function in prokaryotes. Some Cas genes comprise homologous secondary and/or tertiary structures.

crRNA biogenesis in a Type II CRISPR system in nature requires a trans-activating CRISPR RNA (tracrRNA). The tracrRNA is modified by endogenous RNaseIII, and then hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming). The tracrRNA remains hybridized to the crRNA, and the tracrRNA and the crRNA associate with a site-directed polypeptide (e.g., Cas9). The crRNA of the crRNA-tracrRNA-Cas9 complex guides the complex to a target nucleic acid to which the crRNA can hybridize. Hybridization of the crRNA to the target nucleic acid activates Cas9 for targeted nucleic acid cleavage. The target nucleic acid in a Type II CRISPR system is referred to as a protospacer adjacent motif (PAM). In nature, the PAM facilitates binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337(6096):816-821 (2012) showed that the CRISPR/Cas9 system is useful for RNA-programmable genome editing, and International Patent Application Publication Number WO2013/176772 (incorporated herein by reference) provides numerous examples and applications of the CRISPR/Cas endonuclease system for site-specific gene editing.

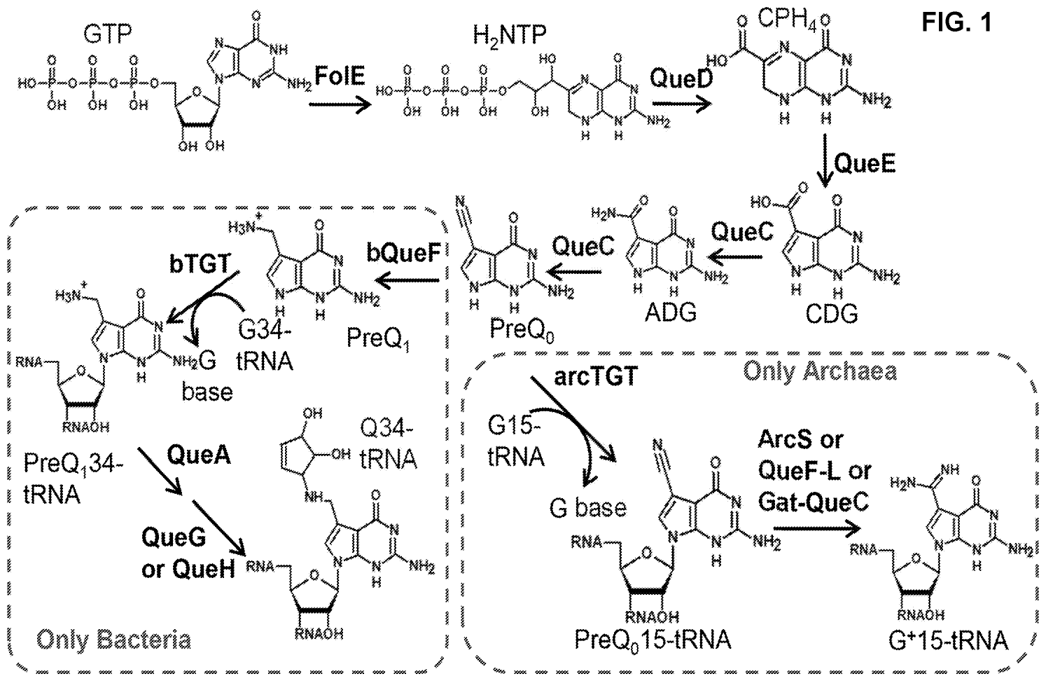

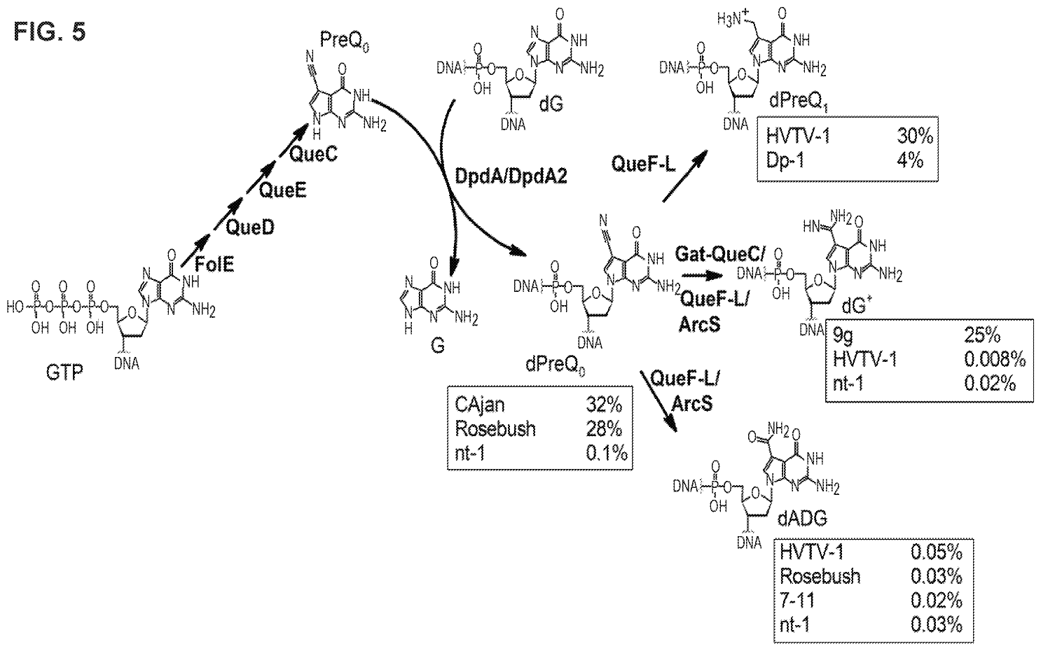

Exemplary CRISPR/Cas polypeptides include the Cas9 polypeptides in FIG. 1 of Fonfara et al., Nucleic Acids Research, 42: 2577-2590 (2014) (incorporated herein by reference). The CRISPR/Cas gene naming system has undergone extensive rewriting since the Cas genes were discovered. FIG. 5 of Fonfara, supra, provides PAM sequences for the Cas9 polypeptides from various species.

Cas9 polypeptides can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining (NHEJ) or alternative non-homologous end joining (A-NHEJ) or microhomology-mediated end joining (MMEJ)). NHEJ can repair cleaved target nucleic acid without the need for a homologous template. This can sometimes result in small deletions or insertions (indels) in the target nucleic acid at the site of cleavage, and can lead to disruption or alteration of gene expression. HDR can occur when a homologous repair template, or exogenous nucleic acid, is available.

Thus, in some embodiments, homologous recombination is used to insert heterologous nucleic acid into the genome of the host bacterium. The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The processes of deleting genomic DNA and integrating non-native nucleic acid into genomic DNA are examples of genome editing.

In some aspects, the Cas9 nuclease is introduced to the bacterium as a protein (i.e., a protein-based system). Typically, the bacteria is treated chemically, electrically, or mechanically to allow Cas9 nuclease entry into the cell. Alternatively, the Cas9 nuclease is introduced to the bacterium as a nucleic acid (e.g., DNA or mRNA) under conditions which allow production of the nuclease. Guide RNA also is introduced into the bacterium.

A genome-targeting RNA is referred to as a “guide RNA” or “gRNA” herein. A guide RNA comprises at least a spacer sequence that hybridizes to a target nucleic acid sequence of interest, and a CRISPR repeat sequence. In Type II systems, the gRNA also comprises a tracrRNA sequence. In the Type II guide RNA, the CRISPR repeat sequence and tracrRNA sequence hybridize to each other to form a duplex. The duplex binds a site-directed polypeptide, such that the guide RNA and site-direct polypeptide form a complex. The guide RNA provides target specificity to the complex by virtue of its association with the Cas9 nuclease. The guide RNA thus directs the activity of the Cas9 nuclease. In some embodiments, the guide RNA is a single molecule guide RNA (sgRNA).

A single-molecule guide RNA in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins.

A nucleic acid encoding the Cas9 nuclease and/or guide RNA is typically delivered in an expression vector. The exogenous nucleic acid can be delivered in the same vector as the Cas9 nucleic acid, or in a second vector. Any of the expression vectors described herein may be used to deliver Cas9 nuclease-encoding nucleic acid into the bacterium. In many aspects, the expression vector is a plasmid. In some embodiments, an expression vector comprises one or more transcription and/or translation control elements. Depending on the host/vector system utilized, any of a number of suitable transcription and translation control elements, including constitutive and inducible promoters, transcription enhancer elements, transcription terminators, etc., may be used.

The Cas9 nuclease-encoding nucleic acid is operably linked to a promoter that drives protein expression. Exemplary prokaryotic promoters include, but are not limited to, wMel WSP Promote, wDc WSP Promoter and T7. For expressing small RNAs, including guide RNAs used in connection with Cas or Cpf1 endonuclease, promoters such as RNA polymerase III promoters, including for example U6 and H1, can be advantageous. Suitable promoters, as well as parameters for enhancing the use of such promoters, are known in art, and additional information and approaches are regularly being described; see, e.g., Ma, H. et al., Molecular Therapy—Nucleic Acids 3, e161 (2014) doi:10.1038/mtna.2014.12.

In various aspects, the heterologous nucleic acid is of bacteriophage origin. Indeed, in some embodiments, the materials and methods described herein are used to efficiently generate stocks of phage for laboratory or therapeutic use. Phages are an attractive therapeutic option for treating bacterial infections, as phages are more specific than antibiotics, are generally harmless to animals and humans, and have been shown to be effective in combatting antibiotic-resistant bacterial infections. Antibiotic-resistant bacterial infections are an increasing concern in clinical and non-clinical settings. Current first-line treatments rely upon the administration of small-molecule antibiotics to induce bacterial cell death. These broad-spectrum treatments disrupt the patient's normal microflora, allowing resistant bacteria and fungal pathogens to take advantage of vacated niches.

In this regard, described herein is method of producing a bacteriophage composition (e.g., a stock of bacteriophage) comprising (a) modifying a nucleic acid of bacteriophage origin to incorporate one or more deazaguanine bases as described herein; (b) introducing the modified nucleic acid into a host bacteria cell; (c) incubating the host bacteria cell until phage-mediated bacterial lysis occurs; and (d) isolating bacteriophage lysate. Optionally, the bacteriophage lysate is purified to produce a pharmaceutical composition of bacteriophage. The bacteriophage may be further modified to produce one or more anti-bacterial toxins.

Any suitable means for culturing bacterial cells is contemplated. Conditions for the culture and production of bacterial cells are readily available and well-known in the art. Cell culture media in general are set forth in Atlas and Parks (eds.) The Handbook of Microbiological Media (1993) CRC Press, Boca Raton, Fla. which is incorporated herein by reference. Additional information for cell culture is found in available commercial literature such as the Life Science Research Cell Culture Catalogue (1998) from Sigma-Aldrich, Inc (St Louis, Mo.) (“Sigma-LSRCCC”) and, for example, The Plant Culture Catalogue and supplement (1997) also from Sigma-Aldrich, Inc (St Louis, Mo.) (“Sigma-PCCS”), all of which are incorporated herein by reference. Also reference is made to the Manual of Industrial Microbiology and Biotechnology. A. Demain and J. Davies Eds. ASM Press. 1999.

In some embodiments, the cell culture medium is a liquid medium. In some embodiments, the cell culture medium is a semi-solid medium (e.g., cultured in semi-solid agar on a plate of solid agar).

In some embodiments, the bacteria (or bacterial cells) are grown under batch or continuous fermentations conditions. Classical batch fermentation is a closed system, wherein the compositions of the medium is set at the beginning of the fermentation and is not subject to artificial alterations during the fermentation. A variation of the batch system is a fed-batch fermentation. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is a system where a defined fermentation medium is added continuously to a bioreactor and an equal amount of conditioned medium (e.g., containing the desired end-products) is removed simultaneously for processing. Continuous fermentation generally maintains the cultures at a constant high density where cells are primarily in the growth phase where production of end products is enhanced. Continuous fermentation systems strive to maintain steady state growth conditions. Methods for modulating nutrients and growth factors for continuous fermentation processes as well as techniques for maximizing the rate of product formation are well known in the art of industrial microbiology.

In some embodiments, the bacteriophage are isolated or purified from the lysate. For example, the culture medium can be filtered through a very small pore size filter to retain the bacteria and permit the smaller bacteriophage to pass through. Typically, a filter having a pore size in the range of from about 0.01 to about 1 μm can be used (or from about 0.1 to about 0.5 μm, or from about 0.2 to about 0.4 μm). Alternatively or in addition, the culture medium is purified from bacterial debris and endotoxins by dialysis using the largest pore membrane that retains bacteriophages, where the membrane preferably has a molecular cut-off of approximately 10⁴ to about 10⁷ daltons (or from about 10⁵ to about 10⁶ daltons). Many other suitable methods can be performed as disclosed for example in US 2001/0026795; US 2002/0001590; U.S. Pat. Nos. 6,121,036; 6,399,097; 6,406,692; 6,423,299; and WO 02/07742, the disclosures of which are incorporated herein by reference in their entireties.

Bacteria (or bacterial cells) for use according to the disclosure include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, Oxalobacter formigenes and Saccharomyces boulardii. In some embodiments, the bacterium is E. coli, B. cereus or L. acidophilus.

In some embodiments, the bacterium is a species of the genus Escherichia (e.g., E. coli). In various embodiments, the E. coli bacterial strain used in the processes described herein are derived from strain W3110, strain MG1655, strain B766 (E. coli W) or strain BW25113.

Other examples of useful E. coli strains include, but are not limited to, E. coli strains found in the E. coli Stock Center from Yale University (at website cgsc.biology.yale.edu/index.php); the Keio Collection, available from the National BioResource Project at NBRP E. coli, Microbial Genetics Laboratory, National Institute of Genetics 1111 Yata, Mishima, Shizuoka, 411-8540 Japan (www at shigen.nig.ac.jp/ecoli/strain/top/top.jsp); or strains deposited at the American Type Culture Collection (ATCC).

The bacteriophage described herein are optionally used to treat a bacterial infection in a subject in need thereof. In this regard, a suitable method comprises administering a bacteriophage comprising a heterologous nucleic acid comprising one or more deazapurine bases to the subject. In some embodiments, the bacterial infection is an Actinobacteria, Aquificae, Armatimonadetes, Bacteroidetes, Caldiserica, Chlamydiae, Chloroflexi, Chrysiogenetes, Cyanobacteria, Deferribacteres, Deinococcus-Thermus, Dictyoglomi, Elusimicrobia, Fibrobacteres, Firmicutes (e.g., Bacillus, Listeria, Staphylococcus), Fusobacteria, Gemmatimonadetes, Nitrospirae, Planctomycetes, Proteobacteria (e.g., Acidobacillus, Aeromonas, Burkholderia, Neisseria, Shewanella, Citrobacter, Enterobacter, Envinia, Escherichia, Klebsiella, Kluyvera, Morganella, Salmonella, Shigella, Yersinia, Coxiella, Rickettsia, Legionella, Avibacterium, Haemophilus, Pasteurella, Acinetobacter, Moraxella, Pseudomonas, Vibrio, Xanthomonas), Spirochaetes, Synergistets, Tenericutes (e.g., Mycoplasma, Spiroplasma, Ureaplasma), Thermodesulfobacteria or a Thermotoga infection. Optionally, the bacteriophage targets Salmonella spp., Listeria monocytogenes, MRSA, E. coli, Mycobacterium tuberculosis, Campylobacter spp., and/or Pseudomonas syringae. Alternatively, the bacteriophage is employed to destroy bacteria ex vivo (e.g., for surface sterilization).

In some embodiments, the heterologous nucleic acid (e.g., heterologous nucleic acid present in bacteriophage) is provided in a pharmaceutical composition, wherein the delivery vehicle is a pharmaceutically acceptable carrier. Pharmaceutically acceptable carriers are well known, and one skilled in the pharmaceutical art can easily select carriers suitable for particular routes of administration (Remington's Pharmaceutical Sciences, Mack Publishing Co., Easton, Pa., 1985). Merely to illustrate, in the context of bacteriophage, the delivery vehicle optionally further stabilizes and/or enhances the efficacy of bacteriophage in inhibiting bacterial infection. In some embodiments, the delivery vehicle is a liquid vehicle suitable for administration by infusion or injection. In some embodiments, the delivery vehicle comprises a buffer. Exemplary buffers include, but are not limited to, phosphate buffered saline (PBS), lysogeny broth (LB), phage buffer (100 mM NaCl, 100 mM Tris-HCl, 0.01% (w/v) Gelatin), and Tryptic Soy broth (TSB). In some embodiments, the delivery vehicle is a solid vehicle suitable for administration, e.g., by inhalation or for application by spraying. In some embodiments, the delivery vehicle is a semi-solid or semi-liquid vehicle, such as a gel, cream, paraffin wax, or ointment, suitable for topical application.

All of the U.S. patents, U.S. patent application publications, U.S. patent applications, foreign patents, foreign patent applications and non-patent publications referred to in this specification, are incorporated herein by reference, in their entireties.

From the foregoing it will be appreciated that, although specific embodiments of the invention have been described herein for purposes of illustration, various modifications may be made without deviating from the spirit and scope of the invention.

### Examples

Materials/Methods

Media composition: Lysogeny broth¹ (LB): 10 g/L tryptone, 5 g/L yeast extract, 10 g/L NaCl, powder order from fisher (BP1426).

Brain heart infusion² (BHI): Merck cat. 110493

BHI+³: BHI supplemented with 8 μM MnCl₂, 0.25 mM, CaCl₂, 0.2 mM MgSO₄, 50 Mm Tris-HCl pH 7.5, 50 ng/μl choline chloride, 0.4% glycine and 100 μl/ml catalase.

Middlebrook 7H9 broth: 4.7 g Middlebrook 7H9 (Difco), 5 mL 40% glycerol, 900 mL ddH2O.

Middlebrook 7H10 agar: 19.0 g Middlebrook 7H10 (Difco), 12.5 mL 40% glycerol, 4.95 mL 40% dextrose, 5 drops anti-bubble, 990 mL ddH2O.

Middlebrook Top Agar: 4.7 g Middlebrook 7H9 (Difco), 7.0 g BactoAgar, ddH2O up to 1000 mL, 4 drops of anti-bubble.

Salt water (SW) stock (30%): 240 g/L NaCl, 30 g/L MgCl₂, 35 g/L MgSO₄, 7 g/L KCl, 5 mM Tris-HCl pH 7.5.

Modified growth medium (Rodrigez-Valera 1983) (MGM): for liquid broth 23% SW is used, 20% for agar medium and 18% for soft-agar medium. 5 g/L peptone and 1 g/L yeast extract are also added.

Difco nutrient broth: 3 g/L beef extract, 5 g/L peptone.

To these media, 15 g/L of agar are uses for solid medium and 7 g/L for top-agar medium.

Construction of the E. coli Q⁻ mutants: The E. coli BW25113 folE::kan, queD::kan, queE::kan, queC::kan and tgt::kan mutants were collected from the Keio collection⁴. Each mutation was transduced using phage P1⁵ in E. coli MG1655. The transductions were verified by PCR (couple of primers used: GO119/GO120 and GO121/GO122 for folE mutation, GO123/GO124 and GO125/GO126 for queD mutation, GO127/GO128 and GO129/GO130 for queE mutation, GO111/GO112 and GO113/GO114 for queC mutation, GO107/GO108 and GO109/GO110 for tgt mutation). The kanamycin cassette was removed from all these strains but Δtgt using pCP20 as described by Datsenko and Wanner⁶. The resulting strains are listed in Table 1.

| Accession # | Name | DpdA | DpdA2 | FolE | QueD |
|---|---|---|---|---|---|
| NC_024146 | Enterobacteria phage 9g | YP_009032326 | | YP_009032327 | YP_009032328 |
| NC_029021 | Enterobacteria phage JenK1 | YP_009219311 | | YP_009219312 | YP_009219313 |
| NC_029028 | Enterobacteria phage JenP1 | YP_009220002 | | YP_009220004 | YP_009220005 |
| NC_028997 | Enterobacteria phage JenP2 | YP_009216970 | | YP_009216971 | YP_009216972 |
| MG948468 | Pantoea phage | AVJ51789 | | AVJ51790 | AVJ51791 |
| | vB_PagS_Vid5 | | | | |
| KX898399 | Pseudomonas phage JG012 | ARB11100 | | ARB11102 | ARB11103 |
| KX898400 | Pseudomonas phage JG054 | ARB11177 | | ARB11178 | ARB11179 |
| NC_031058 | Pseudomonas phage NP1 | YP_009285845 | | YP_009285847 | YP_009285848 |
| JQ067084 | Pseudomonas phage | ALH23795 | | ALH23793 | ALH23791 |
| | PaMx25 | | | | |
| KY926791 | Salmonella phage SE1 | ARM70139 | | ARM70137 | ARM70136 |
| | (in:Nonagvirus) | | | | |
| NC_020860 | Cellulophaga phage phiSM | YP_007675729 | | YP_007675734 | YP_007675726 |
| KY606587 | Dinoroseobacter phage | ARB06140 | | ARB06137 | ARB06133 |
| | vB_DshS-R5C | | | | |
| NC_015274 | Streptococcus phage Dp-1 | YP_004306895 | | YP_004306893 | YP_004306891 |
| KC821610 | Cellulophaga phage | AGO47783 | | AGO47788 | AGO47780 |
| | phi3ST:2 | | | | |
| NC_006268 | Sulfolobus virus STSV1 | YP_077211 | | | |
| JQ287645 | Sulfolobus virus STSV2 | YP_007348259 | | | |
| NC_019515 | Bacillus phage BCD7 | YP_007005872 | | YP_007005873 | YP_007005879 |
| NC_009447 | Burkholderia phage | YP_001210251 | | YP_001210253 | YP_001210254 |
| | BcepGomr | | | | |
| NC_028776 | Escherichia phage CAjan | YP_009196829 | | YP_009196831 | YP_009196832 |
| KX534337 | Escherichia phage Greed | ANY29829 | | ANY29831 | ANY29832 |
| | (partial genome) | | | | |
| NC_027378 | Escherichia phage Seurat | YP_009151977 | | YP_009151979 | YP_009151980 |
| NC_028831 | Escherichia phage slur01 | YP_009201618 | | YP_009201620 | YP_009201621 |
| LT907986 | Escherichia phage | SOE45440 | | | SOE45194 |
| | vB_Eco_SLUR25 | | | | |
| JN699004 | Mycobacterium phage Ares | AER48624 | | AER48628 | AER48626 |
| MH051249 | Mycobacterium phage Boyle | AVR76497 | | AVR76501 | AVR76499 |
| KT880194 | Mycobacterium phage Glass | AMB17316 | | AMB17320 | AMB17318 |
| KR997932 | Mycobacterium phage | AKU45201 | | AKU45205 | AKU45203 |
| | Godines | | | | |
| JN698991 | Mycobacterium phage | AER47234 | | AER47238 | AER47236 |
| | Hedgerow | | | | |
| MG812490 | Mycobacterium phage | AUX82170 | | AUX82174 | AUX82172 |
| | Holeinone | | | | |
| MG812491 | Mycobacterium phage | AUX82212 | | AUX82216 | AUX82214 |
| | ItsyBitsy 1 | | | | |
| MH001452 | Mycobacterium phage | AVO21849 | | AVO21853 | AVO21851 |
| | Kheth | | | | |
| KX443696 | Mycobacterium phage | ANZ52296 | | ANZ52300 | ANZ52298 |
| | Laurie | | | | |
| KM101117 | Mycobacterium phage | AIK68776 | | AIK68780 | AIK68778 |
| | LizLemon | | | | |
| MG757162 | Mycobacterium phage Opia | AVE00290 | | AVE00294 | AVE00292 |
| DQ398048 | Mycobacterium phage | YP_655682 | | YP_655686 | YP_655684 |
| | Qyrzula | | | | |
| AY129334 | Mycobacterium phage | NP_817763 | | NP_817767 | NP_817765 |
| | Rosebush | | | | |
| KT365402 | Mycobacterium phage Tres | ALF01287 | | ALF01291 | ALF01289 |
| KF024722 | Mycobacterium virus Ta17a | AGS81414 | | AGS81418 | AGS81416 |
| NC_027296 | Rhizobium phage RHEph06 | YP_009145910 | | YP_009145913 | YP_009145914 |
| MG962366 | Rhodococcus phage Finch | AVO25132 | | AVO25134 | AVO25133 |
| KY629621 | Streptococcus virus MS1 | AQY55385 | | ARQ32421 | AQY55389 |
| MG592491 | Vibrio phage | AUR88733 | | AUR88734 | AUR88735 |
| | 1.117.O._10N.261.45.E9 | | | | |
| KX198614 | Vibrio phage vB_VhaS-tm | ANO57493 | | ANO57492 | ANO57491 |
| NC_026610 | Vibrio phage VpKK5 | YP_009126591 | | YP_009126590 | YP_009126589 |
| NC_031253 | Mycobacterium phage | YP_009303151 | | YP_009303155 | YP_009303153 |
| | Bipper | | | | |
| MF417872 | Uncultured phage clone | AGH13911 | | AGH13914 | AGH13912 |
| | 7AX_2 (uncomplete | | | | |
| | genome) | | | | |
| KX228400 | Clostridium phage | ANT45221 | | | |
| | CDKM15 | | | | |
| JQ086369 | Enterobacteria phage | YP_007151699 | | | |
| | HK106 | | | | |
| NC_027339 | Enterobacteria phage SfI | YP_009147480 | | | |
| JQ807243 | environmental Halophage | AFH22355 | | | |
| | eHP-23 (partial genome) | | | | |
| JQ807227 | environmental Halophage | AFH21669 | | | |
| | eHP-6 (partial genome) | | | | |
| MG962362 | Mycobacterium phage | AVO24577 | | | |
| | AltPhacts | | | | |
| KX808132 | Mycobacterium phage | APC43635 | | | |
| | Amelie | | | | |
| MF140398 | Mycobacterium phage | ASR86319 | | | |
| | Amohnition | | | | |
| MF377440 | Mycobacterium phage | ASR77972 | | | |
| | Bella96 | | | | |
| NC_028691 | Mycobacterium phage | YP_009191212 | | | |
| | Apizium | | | | |
| MH051264 | Mycobacterium phage | AVR55838 | | | |
| | Cobra | | | | |
| KX683293 | Mycobacterium phage Daffy | AOZ64256 | | | |
| MF140406 | Mycobacterium phage | ASW31785 | | | |
| | DarthP | | | | |
| KX576645 | Mycobacterium phage | AOQ28581 | | | |
| | Derpp | | | | |
| MF919503 | Mycobacterium phage | ATN88731 | | | |
| | Dingo | | | | |
| MF185719 | Mycobacterium phage | ASR85778 | | | |
| | Edugator | | | | |
| NC_028936 | Mycobacterium phage | YP_009211120 | | | |
| | Enkosi | | | | |
| KX576643 | Mycobacterium phage | AOQ28377 | | | |
| | FriarPreacher | | | | |
| MF185720 | Mycobacterium phage | ASR85873 | | | |
| | Guillsminger | | | | |
| KY087993 | Mycobacterium phage | APD18205 | | | |
| | Hammy | | | | |
| JF937095 | Mycobacterium phage | AEK08772 | | | |
| | Harvey | | | | |
| KT364588 | Mycobacterium phage | ALA45631 | | | |
| | Hetaeria | | | | |
| KJ538723 | Mycobacterium phage | AHY84289 | | | |
| | KingVeVeVe | | | | |
| NC_023724 | Mycobacterium phage Larva | YP_009016449 | | | |
| MG962371 | Mycobacterium phage | AVO25553 | | | |
| | LaterM | | | | |
| KX641264 | Mycobacterium phage | AOT25056 | | | |
| | LindNT | | | | |
| MF668276 | Mycobacterium phage | ASZ73467 | | | |
| | Lulumae | | | | |
| NC_026598 | Mycobacterium phage Milly | YP_009125516 | | | |
| NC_028759 | Mycobacterium phage | YP_009195289 | | | |
| | Mufasa | | | | |
| NC_028978 | Mycobacterium phage | YP_009215543 | | | |
| | Murucutumbu | | | | |
| NC_021310 | Mycobacteriumphage | YP_008052097 | | | |
| | Newman | | | | |
| NC_023711 | Mycobacterium phage Oline | YP_009014281 | | | |
| JF704109 | Mycobacterium phage | AEK07224 | | | |
| | Oosterbaan | | | | |
| DQ398046 | Mycobacterium phage Orion | YP 655116 | | | |
| NC_028803 | Mycobacterium phage | YP_009198878 | | | |
| | OSmaximus | | | | |
| KR029086 | Mycobacterium phage | AKF12401 | | | |
| | PDRPv | | | | |
| KR029087 | Mycobacterium phage | AKF12506 | | | |
| | PDRPxv | | | | |
| MF185722 | Mycobacterium phage | ASR85925 | | | |
| | Peanam | | | | |
| NC_028681 | Mycobacterium phage Pops | YP_009189977 | | | |
| KX657794 | Mycobacterium phage | AOZ61371 | | | |
| | SamuelLPlaqson | | | | |
| KC661274 | Mycobacterium phage | AGK87399 | | | |
| | SDcharge11 | | | | |
| KY945355 | Mycobacterium phage | ARQ95482 | | | |
| | Shandong1 | | | | |
| MF919530 | Mycobacterium phage | ATN91769 | | | |
| | Sheila | | | | |
| NC_025438 | Mycobacterium phage Soto | YP_009100829 | | | |
| NC_023563 | Mycobacterium phage | YP_009005667 | | | |
| | Suffolk | | | | |
| NC_028658 | Mycobacterium phage | YP_009187530 | | | |
| | Swish | | | | |
| NC_023498 | Mycobacterium phage | YP_009002693 | | | |
| | Validus | | | | |
| NC_023727 | Mycobacterium phage Vista | YP_009016809 | | | |
| NC_024147 | Mycobacterium phage ZoeJ | YP_009032436 | | | |
| JX042579 | Mycobacterium virus | AFN37736 | | | |
| | MacnCheese | | | | |
| NC_021558 | Paenibacillus phage PG1 | YP_008129913 | | | |
| MG432137 | Pectobacterium phage | ATV25085 | | | |
| | PEAT2 | | | | |
| NC_015938 | Salmonella phage 7-11 | YP_004782407 | | | |
| MG873442 | Salmonella phage SE131 | AVJ48251 | | | |
| NC_029003 | Salmonella phage SEN1 | YP_009217891 | | | |
| NC_019545 | Salmonella phage SPN3UB | YP_007011024 | | | |
| LT714109 | Salmonella virus BTP1 | SIU02687 | | | |
| NC_029046 | Sinorhizobium phage | YP_009221496 | | | |
| | phiLM21 | | | | |
| KX925554 | Streptomyces phage BRock | APC46298 | | | |
| NC_025375 | Stygiolobus rod-shaped | YP_009094239 | | | |
| | virus | | | | |
| NC_004087 | Sulfolobus islandicus rod- | NP_666594 | | | |
| | shaped virus 1 | | | | |
| NC_034625 | Sulfolobus islandicus rod- | YP_009362827 | | | |
| | shaped virus 10 | | | | |
| NC_034624 | Sulfolobus islandicus rod- | YP_009362775 | | | |
| | shaped virus 11 | | | | |
| NC_004086 | Sulfolobus islandicus rod- | NP_666547 | | | |
| | shaped virus 2 | | | | |
| NC_034628 | Sulfolobus islandicus rod- | YP_009362962 | | | |
| | shaped virus 4 | | | | |
| NC_034621 | Sulfolobus islandicus rod- | YP_009362657 | | | |
| | shaped virus 5 | | | | |
| NC_034619 | Sulfolobus islandicus rod- | YP_009362545 | | | |
| | shaped virus 7 | | | | |
| NC_034623 | Sulfolobus islandicus rod- | YP_009362725 | | | |
| | shaped virus 8 | | | | |
| NC_034620 | Sulfolobus islandicus rod- | YP_009362602 | | | |
| | shaped virus 9 | | | | |
| AJ748296 | Sulfolobus islandicus | CAG38826 | | | |
| | rudivirus 1 variant XX | | | | |
| NC_030884 | Sulfolobus islandicus | YP_009272960 | | | |
| | rudivirus 3 | | | | |
| KT997866 | uncultured Mediterranean | ANS03705 | | | |
| | phage uvDeep-CGR2- | | | | |
| | KM23-C246 | | | | |
| MG592472 | Vibrio phage | AUR87363 | | | |
| | 1.100.O._10N.261.45.C3 | | | | |
| MG592540 | Vibrio phage | AUR92499 | | | |
| | 1.173.O._10N.261.55.A11 | | | | |
| MG592572 | Vibrio phage | AUR95132 | | | |
| | 1.201.B._10N.286.55.F1 | | | | |
| MG640035 | Vibrio phage Athenal | AUG84879 | | | |
| KU873925 | Pseudomonas phage pf16 | | AND75003 | AND75004 | AND75007 |
| MG592456 | Vibrio phage | | AUR85871 | AUR85870 | AUR85868 |
| | 1.081.O._10N.286.52.C2 | | | | |
| | (partial genome) | | | | |
| NC_005083 | Vibrio phage KVP40 | | NP_899370 | NP_899369 | NP_899368 |
| NC_028829 | Vibrio phage ValKK3 | | YP_009201241 | YP_009201242 | YP_009201243 |
| NC_023568 | Vibrio phage VH7D | | YP_009006086 | YP_009006085 | YP_009006084 |
| KT919973 | Vibrio phage phi-ST2 | | ALP47368 | ALP47437 | ALP47397 |
| JN849462 | Vibriophage phi-pp2 | | AFN37353 | AFN37351 | AFN37350 |
| NC_021529 | Vibrio phage nt-1 | | YP_008125322 | YP_008125321 | YP_008125319 |
| KR560069 | Stenotrophomonas phage | | AKO61693 | AKO61689 | AKO61579 |
| | IME-SM1 | | | | |
| KY979132 | Acidovorax phage ACP17 | | ASD50403 | ASD50401 | ASD50399 |
| NC_021330 | Halovirus HCTV-1 | | | YP_008059626 | YP_008059634 |
| NC_021327 | Halovirus HCTV-5 | | | YP_008059110 | YP_008059116 |
| NC_020158 | Halovirus HVTV-1 | | | YP_007378975 | YP_007378981 |
| NC_019507 | Campylobacter phage CP21 | | | YP_007005301 | YP_007005116 |
| NC_027997 | Campylobacter phage | | | YP_009169258 | YP_009169203 |
| | CP220 | | | | |
| NC_027996 | Campylobacter phage CPt10 | | | YP_009169065 | YP_009169010 |
| HM246724 | Campylobacter phage | | | AEI88255 | AEF56764 |
| | IBB35 | | | | |
| LT598654 | Phage NCTB | | | SBV38459 | SBV38375 |
| KY487993 | Uncultured virus clone | | | ASF00408 | ASF00598 |
| | CG99 | | | | |
| NC_592671 | Vibrio phage | | | AUS03055 | AUS03064 |
| | 2.275.O._10N.286.54.E11 | | | | |
| NC_021803 | Cellulophaga phage phi13:1 | | | AGO49043 | AGO49041 |
| KC821604 | Cellulophaga phage phiST | | | AGO47177 | AGO47175 |
| KT588073 | Acinetobacter phage Ab105- | | | ALJ98956 | |
| | 3phi | | | | |
| NC_021858 | Pandoravirus dulcis | | | YP_008318610 | |
| NC_026440 | Pandoravirus inopinatum | | | YP_009120778 | |
| NC_022098 | Pandoravirus salinus | | | YP_008436542 | |
| NC_031245 | Bacillus phage SP-15 | | | | |
| LC373201 | Enterobacter phage phiEM4 | | | | |
| NC_027340 | Erwinia phage phiEa2809 | | | | |
| NC_025446 | Escherichia phage ECML-4 | | | | |
| MG383452 | Escherichia phage FEC14 | | | | |
| NC_019452 | Escherichia phage PhaxI | | | | |
| JN593240 | Escherichia virus CBA120 | | | | |
| NC_022343 | Klebsiella phage 0507-KN2-1 | | | | |
| MG428990 | Klebsiella phage Menlow | | | | |
| NC_023744 | Mycobacterium phage | | | | |
| | DS6A | | | | |
| NC_022054 | Mycobacterium phage | | | | |
| | Muddy | | | | |
| NC_021063 | Mycobacterium phage | | | | |
| | vB_MapS_FF47 | | | | |
| MF063068 | Pseudomonas phage Noxifer | | | | |
| NC_028899 | Ralstonia phage RSF1 | | | | |
| AP014693 | Ralstonia phage RSL2 | | | | |
| JX006077 | Saccharomonospora phage | | | | |
| | PIS 136 | | | | |
| NC_029042 | Salmonella phage 38 | | | | |
| NC_031045 | Salmonella phage GG32 | | | | |
| NC_019530 | Salmonella phage PhiSH19 | | | | |
| NC_016073 | Salmonella phage SFP10 | | | | |
| JX0081828 | Salmonella phage STML- | | | | |
| | 13-1 (partial genome) | | | | |
| NC_031128 | Salmonella phage | | | | |
| | vB_SalM_PM10 | | | | |
| NC_023856 | Salmonella phage | | | | |
| | vB_SalM_SJ2 | | | | |
| KX171211 | Salmonella phage | | | | |
| | vB_SenM-2 | | | | |
| NC_015296 | Salmonella phage Vi01 | | | | |
| MF285619 | Serratia phage 2050H1 | | | | |
| NC_020083 | Serratia phage phiMAM1 | | | | |
| KX147096 | Serratia phage | | | | |
| | vB_Sru_IME250 | | | | |
| MG592536 | Vibrio phage | | | | |
| | 1.169.O._10N.261.52.B1 | | | | |
| | (partial genome) | | | | |
| MG592554 | Vibrio phage | | | | |
| | 1.188.A._10N.286.51.A6 | | | | |
| | (partial genome) | | | | |
| MG592609 | Vibrio phage | | | | |
| | 1.244.A._10N.261.54.C3 | | | | |
| KY499642 | Vibrio phage pVa-21 | | | | |
| JQ807233 | environmental Halophage | | | | |
| | eHP-12 | | | | |

| Accession # | Name | QueE | QueC | YhhQ |
|---|---|---|---|---|
| NC_024146 | Enterobacteria phage 9g | YP_009032331 | | |
| NC_029021 | Enterobacteria phage JenK1 | YP_009219316 | | |
| NC_029028 | Enterobacteria phage JenP1 | YP_009220008 | | |
| NC_028997 | Enterobacteria phage JenP2 | YP_009216975 | | |
| MG948468 | Pantoea phage | AVJ51795 | | AVJ51796 |
| | vB_PagS_Vid5 | | | |
| KX898399 | Pseudomonas phage JG012 | ARB11105 | | |
| KX898400 | Pseudomonas phage JG054 | ARB11181 | | |
| NC_031058 | Pseudomonas phage NP1 | YP_009285850 | | |
| JQ067084 | Pseudomonas phage | ALH23789 | | |
| | PaMx25 | | | |
| KY926791 | Salmonella phage SE1 | ARM70133 | | |
| | (in:Nonagvirus) | | | |
| NC_020860 | Cellulophaga phage phiSM | YP_007675725 | YP_007675727 | |
| KY606587 | Dinoroseobacter phage | ARB06149 | ARB06136 | |
| | vB_DshS-R5C | | | |
| NC_015274 | Streptococcus phage Dp-1 | YP_004306892 | YP_004306890 | |
| KC821610 | Cellulophaga phage | AGO47779 | AGO47781 | |
| | phi3ST:2 | | | |
| NC_006268 | Sulfolobus virus STSV1 | | | |
| JQ287645 | Sulfolobus virus STSV2 | | | |
| NC_019515 | Bacillus phage BCD7 | YP_007005878 | YP_007005876 | |
| NC_009447 | Burkholderia phage | YP_001210257 | YP_001210255 | |
| | BcepGomr | | | |
| NC_028776 | Escherichia phage CAjan | YP_009196839 | YP_009196836 | |
| KX534337 | Escherichia phage Greed | ANY29839 | ANY29835 | |
| | (partial genome) | | | |
| NC_027378 | Escherichia phage Seurat | YP_009151987 | YP_009151983 | |
| NC_028831 | Escherichia phage slur01 | YP_009201628 | YP_009201624 | |
| LT907986 | Escherichia phage | SOE45212 | SOE45202 | |
| | vB_Eco_SLUR25 | | | |
| JN699004 | Mycobacterium phage Ares | AER48627 | AER48625 | |
| MH051249 | Mycobacterium phage Boyle | AVR76500 | AVR76498 | |
| KT880194 | Mycobacterium phage Glass | AMB17319 | AMB17317 | |
| KR997932 | Mycobacterium phage | AKU45204 | AKU45202 | |
| | Godines | | | |
| JN698991 | Mycobacterium phage | AER47237 | AER47235 | |
| | Hedgerow | | | |
| MG812490 | Mycobacterium phage | AUX82173 | AUX82171 | |
| | Holeinone | | | |
| MG812491 | Mycobacterium phage | AUX82215 | AUX82213 | |
| | ItsyBitsy 1 | | | |
| MH001452 | Mycobacterium phage | AVO21852 | AVO21850 | |
| | Kheth | | | |
| KX443696 | Mycobacterium phage | ANZ52299 | ANZ52297 | |
| | Laurie | | | |
| KM101117 | Mycobacterium phage | AIK68779 | AIK68777 | |
| | LizLemon | | | |
| MG757162 | Mycobacterium phage Opia | AVE00293 | AVE00291 | |
| DQ398048 | Mycobacterium phage | YP_655685 | YP_655683 | |
| | Qyrzula | | | |
| AY129334 | Mycobacterium phage | NP_817766 | NP_817764 | |
| | Rosebush | | | |
| KT365402 | Mycobacterium phage Tres | ALF01290 | ALF01288 | |
| KF024722 | Mycobacterium virus Ta17a | AGS81417 | AGS81415 | |
| NC_027296 | Rhizobium phage RHEph06 | YP_009145916 | YP_009145915 | |
| MG962366 | Rhodococcus phage Finch | AVO25170 | AVO25169 | |
| KY629621 | Streptococcus virus MS1 | AQY55388 | AQY55390 | ARQ32422 |
| MG592491 | Vibrio phage | AUR88737 | AUR88736 | |
| | 1.117.O._10N.261.45.E9 | | | |
| KX198614 | Vibrio phage vB_VhaS-tm | ANO57488 | ANO57489 | |
| NC_026610 | Vibrio phage VpKK5 | YP_009126587 | YP_009126588 | |
| NC_031253 | Mycobacterium phage | YP_009303154 | | |
| | Bipper | | | |
| MF417872 | Uncultured phage clone | AGH13913 | | |
| | 7AX_2 (uncomplete | | | |
| | genome) | | | |
| KX228400 | Clostridium phage | | | ANT45222 |
| | CDKM15 | | | |
| JQ086369 | Enterobacteria phage | | | |
| | HK106 | | | |
| NC_027339 | Enterobacteria phage SfI | | | |
| JQ807243 | environmental Halophage | | | |
| | eHP-23 (partial genome) | | | |
| JQ807227 | environmental Halophage | | | AFH21670 |
| | eHP-6 (partial genome) | | | |
| MG962362 | Mycobacterium phage | | | |
| | AltPhacts | | | |
| KX808132 | Mycobacterium phage | | | |
| | Amelie | | | |
| MF140398 | Mycobacterium phage | | | |
| | Amohnition | | | |
| MF377440 | Mycobacterium phage | | | |
| | Bella96 | | | |
| NC_028691 | Mycobacterium phage | | | |
| | Apizium | | | |
| MH051264 | Mycobacterium phage | | | |
| | Cobra | | | |
| KX683293 | Mycobacterium phage Daffy | | | |
| MF140406 | Mycobacterium phage | | | |
| | DarthP | | | |
| KX576645 | Mycobacterium phage | | | |
| | Derpp | | | |
| MF919503 | Mycobacterium phage | | | |
| | Dingo | | | |
| MF185719 | Mycobacterium phage | | | |
| | Edugator | | | |
| NC_028936 | Mycobacterium phage | | | |
| | Enkosi | | | |
| KX576643 | Mycobacterium phage | | | |
| | FriarPreacher | | | |
| MF185720 | Mycobacterium phage | | | |
| | Guillsminger | | | |
| KY087993 | Mycobacterium phage | | | |
| | Hammy | | | |
| JF937095 | Mycobacterium phage | | | |
| | Harvey | | | |
| KT364588 | Mycobacterium phage | | | |
| | Hetaeria | | | |
| KJ538723 | Mycobacterium phage | | | |
| | KingVeVeVe | | | |
| NC_023724 | Mycobacterium phage Larva | | | |
| MG962371 | Mycobacterium phage | | | |
| | LaterM | | | |
| KX641264 | Mycobacterium phage | | | |
| | LindNT | | | |
| MF668276 | Mycobacterium phage | | | |
| | Lulumae | | | |
| NC_026598 | Mycobacterium phage Milly | | | |
| NC_028759 | Mycobacterium phage | | | |
| | Mufasa | | | |
| NC_028978 | Mycobacterium phage | | | |
| | Murucutumbu | | | |
| NC_021310 | Mycobacteriumphage | | | |
| | Newman | | | |
| NC_023711 | Mycobacterium phage Oline | | | |
| JF704109 | Mycobacterium phage | | | |
| | Oosterbaan | | | |
| DQ398046 | Mycobacterium phage Orion | | | |
| NC_028803 | Mycobacterium phage | | | |
| | OSmaximus | | | |
| KR029086 | Mycobacterium phage | | | |
| | PDRPv | | | |
| KR029087 | Mycobacterium phage | | | |
| | PDRPxv | | | |
| MF185722 | Mycobacterium phage | | | |
| | Peanam | | | |
| NC_028681 | Mycobacterium phage Pops | | | |
| KX657794 | Mycobacterium phage | | | |
| | SamuelLPlaqson | | | |
| KC661274 | Mycobacterium phage | | | |
| | SDcharge11 | | | |
| KY945355 | Mycobacterium phage | | | |
| | Shandong1 | | | |
| MF919530 | Mycobacterium phage | | | |
| | Sheila | | | |
| NC_025438 | Mycobacterium phage Soto | | | |
| NC_023563 | Mycobacterium phage | | | |
| | Suffolk | | | |
| NC_028658 | Mycobacterium phage | | | |
| | Swish | | | |
| NC_023498 | Mycobacterium phage | | | |
| | Validus | | | |
| NC_023727 | Mycobacterium phage Vista | | | |
| NC_024147 | Mycobacterium phage ZoeJ | | | |
| JX042579 | Mycobacterium virus | | | |
| | MacnCheese | | | |
| NC_021558 | Paenibacillus phage PG1 | | | |
| MG432137 | Pectobacterium phage | | | |
| | PEAT2 | | | |
| NC_015938 | Salmonella phage 7-11 | | | |
| MG873442 | Salmonella phage SE131 | | | |
| NC_029003 | Salmonella phage SEN1 | | | |
| NC_019545 | Salmonella phage SPN3UB | | | |
| LT714109 | Salmonella virus BTP1 | | | |
| NC_029046 | Sinorhizobium phage | | | YP_009221497 |
| | phiLM21 | | | |
| KX925554 | Streptomyces phage BRock | | | |
| NC_025375 | Stygiolobus rod-shaped | | | |
| | virus | | | |
| NC_004087 | Sulfolobus islandicus rod- | | | |
| | shaped virus 1 | | | |
| NC_034625 | Sulfolobus islandicus rod- | | | |
| | shaped virus 10 | | | |
| NC_034624 | Sulfolobus islandicus rod- | | | |
| | shaped virus 11 | | | |
| NC_004086 | Sulfolobus islandicus rod- | | | |
| | shaped virus 2 | | | |
| NC_034628 | Sulfolobus islandicus rod- | | | |
| | shaped virus 4 | | | |
| NC_034621 | Sulfolobus islandicus rod- | | | |
| | shaped virus 5 | | | |
| NC_034619 | Sulfolobus islandicus rod- | | | |
| | shaped virus 7 | | | |
| NC_034623 | Sulfolobus islandicus rod- | | | |
| | shaped virus 8 | | | |
| NC_034620 | Sulfolobus islandicus rod- | | | |
| | shaped virus 9 | | | |
| AJ748296 | Sulfolobus islandicus | | | |
| | rudivirus 1 variant XX | | | |
| NC_030884 | Sulfolobus islandicus | | | |
| | rudivirus 3 | | | |
| KT997866 | uncultured Mediterranean | | | |
| | phage uvDeep-CGR2- | | | |
| | KM23-C246 | | | |
| MG592472 | Vibrio phage | | | |
| | 1.100.O._10N.261.45.C3 | | | |
| MG592540 | Vibrio phage | | | |
| | 1.173.O._10N.261.55.A11 | | | |
| MG592572 | Vibrio phage | | | |
| | 1.201.B._10N.286.55.F1 | | | |
| MG640035 | Vibrio phage Athenal | | | |
| KU873925 | Pseudomonas phage pf16 | AND75009 | AND75001 | |
| MG592456 | Vibrio phage | AUR86020 | AUR85990 | |
| | 1.081.O._10N.286.52.C2 | | | |
| | (partial genome) | | | |
| NC_005083 | Vibrio phage KVP40 | NP_899531 | NP_899504 | |
| NC_028829 | Vibrio phage ValKK3 | YP_009201467 | YP_009201239 | |
| NC_023568 | Vibrio phage VH7D | YP_009006244 | YP_009006088 | |
| KT919973 | Vibrio phage phi-ST2 | ALP47407 | ALP47426 | |
| JN849462 | Vibriophage phi-pp2 | AFN37515 | AFN37486 | |
| NC_021529 | Vibrio phage nt-1 | YP_008125479 | YP_008125323 | |
| KR560069 | Stenotrophomonas phage | AKO61694 | AKO61692 | |
| | IME-SM1 | | | |
| KY979132 | Acidovorax phage ACP17 | ASD50406 | | |
| NC_021330 | Halovirus HCTV-1 | YP_008059630 | YP_008059635 | YP_008059627 |
| NC_021327 | Halovirus HCTV-5 | YP_008059113 | YP_008059117 | |
| NC_020158 | Halovirus HVTV-1 | YP_007378978 | YP_007378982 | |
| NC_019507 | Campylobacter phage CP21 | YP_007005215 | YP_007005322 | |
| NC_027997 | Campylobacter phage | YP_009169151 | YP_009169248 | |
| | CP220 | | | |
| NC_027996 | Campylobacter phage CPt10 | YP_009168954 | YP_009169054 | |
| HM246724 | Campylobacter phage | AEI88211 | AEI88267 | |
| | IBB35 | | | |
| LT598654 | Phage NCTB | SBV38478 | SBV38455 | |
| KY487993 | Uncultured virus clone | | ASF00594 | |
| | CG99 | | | |
| NC_592671 | Vibrio phage | AUS03059 | AUS03053 | AUS03052 |
| | 2.275.O._10N.286.54.E11 | | | |
| NC_021803 | Cellulophaga phage phi13:1 | AGO49042 | | |
| KC821604 | Cellulophaga phage phiST | AGO47176 | | |
| KT588073 | Acinetobacter phage Ab105- | | | |
| | 3phi | | | |
| NC_021858 | Pandoravirus dulcis | | | |
| NC_026440 | Pandoravirus inopinatum | | | |
| NC_022098 | Pandoravirus salinus | | | |
| NC_031245 | Bacillus phage SP-15 | | YP_009302501 | |
| LC373201 | Enterobacter phage phiEM4 | | BBD52218 | |
| NC_027340 | Erwinia phage phiEa2809 | | YP_009147529 | |
| NC_025446 | Escherichia phage ECML-4 | | YP_009101458 | |
| MG383452 | Escherichia phage FEC14 | | ATW66911 | |
| NC_019452 | Escherichia phage PhaxI | | YP_007002664 | |
| JN593240 | Escherichia virus CBA120 | | YP_004957727 | |
| NC_022343 | Klebsiella phage 0507-KN2-1 | | YP_008532008 | |
| MG428990 | Klebsiella phage Menlow | | AUG87902 | |
| NC_023744 | Mycobacterium phage | | YP_009018690 | |
| | DS6A | | | |
| NC_022054 | Mycobacterium phage | | YP_008408902 | |
| | Muddy | | | |
| NC_021063 | Mycobacterium phage | | YP_007869941 | |
| | vB_MapS_FF47 | | | |
| MF063068 | Pseudomonas phage Noxifer | | ARV77198 | |
| NC_028899 | Ralstonia phage RSF1 | | YP_009207957 | |
| AP014693 | Ralstonia phage RSL2 | | YP_009212990 | |
| JX006077 | Saccharomonospora phage | | AFM10404 | |
| | PIS 136 | | | |
| NC_029042 | Salmonella phage 38 | | YP_009220980 | |
| NC_031045 | Salmonella phage GG32 | | YP_009283840 | |
| NC_019530 | Salmonella phage PhiSH19 | | YP_007007999 | |
| NC_016073 | Salmonella phage SFP10 | | YP_004895194 | |
| JX0081828 | Salmonella phage STML- | | AFU64331 | |
| | 13-1 (partial genome) | | | |
| NC_031128 | Salmonella phage | | YP_009293427 | |
| | vB_SalM_PM10 | | | |
| NC_023856 | Salmonella phage | | YP_009021339 | |
| | vB_SalM_SJ2 | | | |
| KX171211 | Salmonella phage | | ANT44593 | |
| | vB_SenM-2 | | | |
| NC_015296 | Salmonella phage Vi01 | | YP_004327394 | |
| MF285619 | Serratia phage 2050H1 | | ASZ78955 | |
| NC_020083 | Serratia phage phiMAM1 | | YP_007349154 | |
| KX147096 | Serratia phage | | ANM47156 | |
| | vB_Sru_IME250 | | | |
| MG592536 | Vibrio phage | | AUR92104 | |
| | 1.169.O._10N.261.52.B1 | | | |
| | (partial genome) | | | |
| MG592554 | Vibrio phage | | AUR93611 | |
| | 1.188.A._10N.286.51.A6 | | | |
| | (partial genome) | | | |
| MG592609 | Vibrio phage | | AUR97812 | |
| | 1.244.A._10N.261.54.C3 | | | |
| KY499642 | Vibrio phage pVa-21 | | AQT27978 | |
| JQ807233 | environmental Halophage | | | AFH21913 |
| | eHP-12 | | | |

Cloning E. coli tgt: The tgt gene was amplified by PCR from E. coli MG1655 using tgt_pBAD24 KpnI_F and tgt_pBAD24_SphI_R primers. The resulting PCR product and pBAD24 were digested by KpnI and SphI. (NEB) following the recommendation of the manufacturer. The genes were then inserted by ligation using the T4 DNA ligase from NEB, following the manufacturer recommendations. The resulting plasmid was verified by sequencing (data not shown).

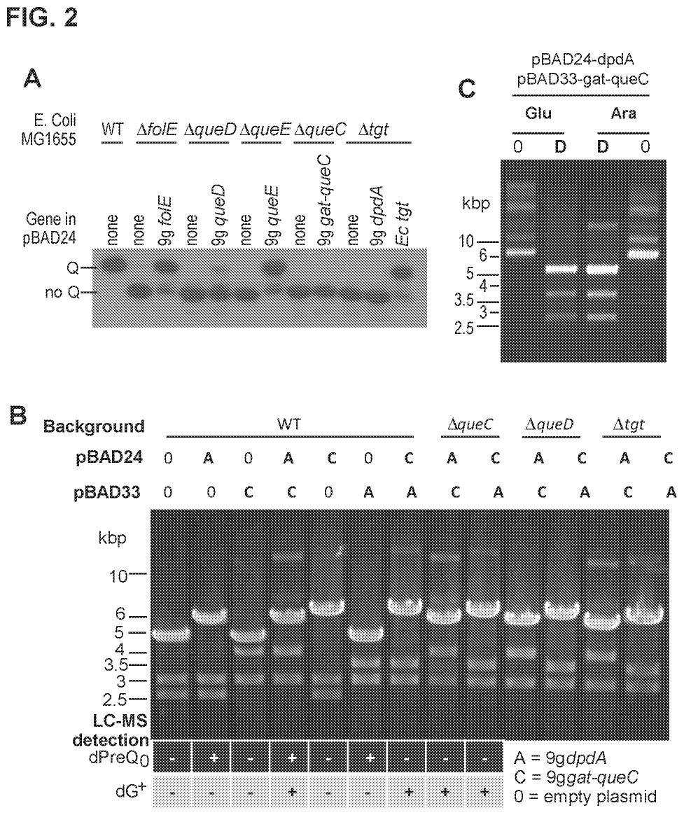

Cloning of 9g genes: dpdA, folE, queD, queE and gat-queC genes from Enterobacteria phage 9g (accession number: NC_024146) were amplified by PCR using the couple of primers GO80/GO81, GO92/GO93, GO94/GO95, GO100/GO101 and GO96/GO97, respectively. pBAD24 plasmid and the PCR products were digested by SalI-HF and SbfI-HF (NEB), following the recommendation of the manufacturer. The genes were then inserted by ligation using the T4 DNA ligase from NEB, following the manufacturer recommendations. dpdA and gat-queC were also cloned in pBAD33 using the same methods. The resulting plasmids were verified by sequencing (data not shown). Each resulting plasmid was transformed in different mutants of E. coli MG1655 as listed in Table 1 for the experiment showed in FIG. 2A. Different couple of plasmids were co transformed in E. coli MG1655, E. coli MG1655 ΔqueC, E. coli MG1655 ΔqueD or E. coli MG1655 Δtgt as listed in Table 1 for the experiment showed in FIG. 2B.

Plasmid DNA preparation for Mass spectrometry: Overnight cultures were diluted 1/100-fold into 500 mL of LB supplemented with 0.4% arabinose, 100 μg/mL ampicillin and 20 μg/mL of chloramphenicol. Cells were grown overnight and pelleted. The Qiagen maxi-prep kit was used to extract the plasmid following the recommendations of the manufacturer.

Rosebush and Orion DNA purification: Mycobacteriophages and Rosebush and Orion were grown as described previously¹³. In brief, 30 mL of a dense M. smegmatis culture was mixed with approximately 106 phage particle, 270 mL of top-agar were added and the mixture was plated on 30 large (150×10 mm) solid media plates. After incubation for 36-48 h at 37° C., 10 mls of phage buffer added, incubated for 4 hrs at room temperature, and the phage lysate collected. Following clarification by centrifugation, phage particles were precipitated with the addition of NaCl to a final concentration of 1M and polyethylene glycol 8000 to a final concentration of 10%. The precipitated particles were collected by centrifugation for 10 minutes at 5,500×g at 4° C., and resuspended in 10 mls of phage buffer. The lysate was clarified by centrifuged at 5,500×g for 10 minutes at 4° C., 8.5 g of CsCl was added, and placed in a heat-sealed tube. Samples were centrifuged at 38,000 RPM (98,000×g) for 16 hours, and the visible phage band removed with a syringe through the side of the tube.

Prior to DNA extraction, CsCl was removed by dialysis against phage buffer overnight at 4° C. For DNA extraction, 0.5 mls of phage lysate (˜1012 particles) were incubated with 12.5 mM MgCl₂, 0.8 μU/mL DNAse I and 100 μg/mL RNAse at room temperature for 30 minutes. To this, 20 mM EDTA, 50 μg/mL of Proteinase K and 0.5% of SDS were added, vortexed vigorously and incubated at 55° C. for 60 minutes. An equal volume of phenol:chlorophorm:isoamyl-alcohol (25:24:1) was added and the mixture was inverted several time before being centrifuged for 5 minutes at room temperature at 13,000 rpm (16,000×g). This step was repeated several times on the aqueous phase obtained until the white interphase was gone. The DNA was ethanol precipitated from the sample, pelleted, washed with 500 μL of 70% ethanol, dried, and the DNA pellet resuspended in 50 μL ddH2O. DNA concentrations were measured using NanoDrop (ThermoScientific).

HVTV-1 DNA purification: To 30 mL of a stationary phase Haloarcula valismoris grown in MGM 23%, enough phages were added to obtain confluent lysis on plates. 270 mL of MGM 18% top-agar were added and the mixture was completely plated on MGM 20% agar. The phages were grown for 4-5 days at 37° C. then a top layer of HVTV-1 virus buffer¹⁴ (1.2 M NaCl, 44 mM MgCl₂, 47 mM MgSO₄, 1.5 mM CaCl₂, 28 mM KCl, 24 mM Tris-HCl pH 7.2) was poured on top of each plate. Phages were allowed to diffuse to the liquid phase for 4 h at 4° C. before being harvested. Debris were pelleted, and phages were precipitated over night at 4° C. by adding 10% polyethylene glycol (PEG 8000) to the supernatant. The phage suspension was centrifuged for 10 minutes at 4,500×g at 4° C. The phage pellet was resuspended in 10 mL of HVTV-1 virus buffer and dialyzed in the same buffer over night at 4° C. to eliminate the last traces of PEG. 12.5 mM MgCl₂, 0.8 μU/mL DNAse I and 100 μg/mL RNAse were added and the mixture were incubated at room temperature for ˜30 minutes. 20 mM EDTA, 50 μg/mL of Proteinase K and 0.5% of SDS were added to the mixture, which was then vortexed vigorously and incubate at 55° C. for 60 minutes. A equal volume of phenol:chlorophorm:isoamyl-alcohol (25:24:1) was then added and the mixture was inverted several time before being centrifuged for 5 minutes at room temperature at 4,500×g. This step was repeated several times on the aqueous phase obtained until the white interphase was gone. An equal volume of chloroform was added to the aqueous phase, vortexed and centrifuged again to eliminate the last traces of phenol. The DNA was then ethanol precipitated from the sample and pelleted. The pellet was washed with 500 μL of 70% ethanol. The dried DNA pellet was then resuspended in ˜50 μL dH₂O. Concentrations were measured using a NanoDrop® ND-1000 Spectrophotometer (Thermo scientific, Waltham, Mass.).

9g DNA purification: To 30 mL of a stationary phase E. coli MG1655 grown in LB, enough phages were added to obtain confluent lysis on plates. 270 mL of LB top-agar were added and the mixture was completely plated on LB agar. The phages were grown overnight at 37° C. then a top layer of TM buffer (10 mM MgSO₄, 10 mM Tris-HCl pH 7.5) was poured on top of each plate. Phages were allowed to diffuse to the liquid phase for 4 h at 4° C. before being harvested. Debris were pelleted, and phages were precipitated over night at 4° C. by adding 1 M of NaCl and 10% polyethylene glycol (PEG 8000) to the supernatant. The phage suspension was centrifuged for 10 minutes at 4,500×g at 4° C. The phage pellet was resuspended in 10 mL of TM buffer and dialyzed in the same buffer over night at 4° C. to eliminate the last traces of PEG. 12.5 mM MgCl₂, 0.8 μU/mL DNAse I and 100 μg/mL RNAse were added and the mixture were incubated at room temperature for ˜30 minutes. 20 mM EDTA, 50 μg/mL of Proteinase K and 0.5% of SDS were added to the mixture, which was then vortexed vigorously and incubate at 55° C. for 60 minutes. A equal volume of phenol:chlorophorm:isoamyl-alcohol (25:24:1) was then added and the mixture was inverted several time before being centrifuged for 5 minutes at room temperature at 4,500×g. This step was repeated several times on the aqueous phase obtained until the white interphase was gone. An equal volume of chloroform was added to the aqueous phase, vortexed and centrifuged again to eliminate the last traces of phenol. The DNA was then ethanol precipitated from the sample and pelleted. The pellet was washed with 500 μL of 70% ethanol. The dried DNA pellet was then resuspended in ˜50 μL dH₂O. Concentrations were measured using a NanoDrop® ND-1000 Spectrophotometer (Thermo scientific, Waltham, Mass.).

### Synthesis of 2-Amino-7-(2-deoxy-β-D-erythro-pentofuranosyl)-4,7-dihydro-4-oxo-1H-pyrrolo[2,3-d]pyrimidine-5-carboxamide (dADG)

To a solution of compound i¹⁶ (130 mg, 0.33 mmol, FIG. 7) in 1:1 MeOH-dioxane (12 mL) was added Et₃N (0.2 mL, 1.5 mmol) and purged with CO gas for 10 min followed by addition of Pd(PhCN)₂Cl₂ (12.7 mg, 0.03 mmol). The reaction mixture was stirred at 60° C. for 24 h, cooled to ambient temperature and evaporated. To the resulting crude ester was added aqueous ammonia (15 mL) in a sealed tube, which was heated at 100° C. for 1 h. The reaction mixture was cooled to ambient temperature and evaporated to dryness. The crude reaction mixture was washed with hot methanol to afford dADG (60 mg, 58%) as off-white solid. FIRMS (ESI): m/z calculated for C₁₂H₁₆N₅O₅ [M+H]⁺ 310.1151, observed 310.1152.

### Synthesis of 2-amino-7-(2-deoxy-β-D-erythro-pentofuranosyl)-4,7-dihydro-4-oxo-3H-pyrrolo[2,3-d]pyrimidine-5-carbonitrile (dPreQ₀)¹⁷

To a suspension of i¹⁶ (600 mg, 1.53 mmol) in pyridine (10 mL) was added CuCN (1.37 g, 15.3 mmol) with stirring under reflux for 20 h. The reaction mixture was cooled to ambient temperature and solvent evaporated. The resulting solid was washed thoroughly with 20% MeOH in dichloromethane, with the washings combined, evaporated and purified by column chromatography (100-200 mesh silica gel) eluting with 10% to 20% MeOH in dichloromethane to afford dPreq₀ (220 mg, 49%) as off-white solid. HRMS (ESI): m/z calculated for C₁₂H₁₄N₅O₄ [M+H]⁺ 292.1046, observed 292.1043.

### Synthesis of 2-Amino-7-(2-deoxy-β-D-erythro-pentofuranosyl)-4,7-dihydro-4-oxo-3H-pyrrolo[2,3-d]pyrimidine-5-carboximidamide (dG⁺)

Dry HCl gas was bubbled through a suspension of dPreQ₀ (100 mg, 0.34 mmol) in anhydrous MeOH (20 mL) at 0° C. for 2 h. Following stirring at ambient temperature for 16 h, the reaction mixture was evaporated and treated with 7N NH₃ in MeOH at 0° C., with stirring for 16 h. The crude reaction mixture was evaporated under vacuum and purified by MPLC using C18 column eluting with acetonitrile and H₂O. The fractions containing product was lyophilized to afford dG⁺ (20 mg, 18%) as an off-white solid¹⁸. HRMS (ESI): m/z calculated for C₁₂H₁₇N₆O₄ [M+H]⁺ 309.1311, observed 309.1306.

Q detection in tRNA: Overnight cultures were diluted 1/100-fold into 5 mL of LB supplemented with 0.4% arabinose and 100 μg/mL ampicillin and grown for 2 h at 37° C. Cells were harvested by centrifugation at 16,000×g for 2 min at 4° C. Cell pellets were immediately resuspended in 1 mL of Trizol (Life technologies, Carlsbad, Calif.). Small RNAs were extracted using PureLink™ miRNA Isolation kit from Invitrogen (Carlsbad, Calif.) according to manufacturer protocol. The purified RNAs were eluted in 50 μL of RNase free water and tRNA concentrations were measured by NanoDrop® ND-1000 Spectrophotometer (Thermo scientific, Waltham, Mass.). Then, 200 μs were used in 3-(Acrylamido)-phenylboronic acid (APB) assay described in detail previously³² using the (5′-biotin-CCCTCGGTGACAGGCAGG-3′) probe that detects tRNA_{Asp}(GUC) at final concentration of 0.3 μM.

Restriction assay for deazapurine presence in plasmid DNA: E. coli strains containing different variation of pBAD24 and pBAD33 (with or without dpdA or gat-queC from Enterobacteria phage 9g) were grown overnight in LB supplemented with 0.2% of glucose at 37° C. Each strain was diluted 100-fold in LB supplemented with 0.4% of arabinose and grown 6 h at 37° C. Plasmids were extracted using the Qiagen QIAprep Spin Miniprep Kit and 500 ng of plasmid were digested by EcoRI-HF (New England Biolabs, Ipswich Mass.) for 1 h at 37° C. in 20 mL CutSmart buffer. The enzyme was inactivated by 20 min incubation at 80° C. The samples were run on a 0.5% agarose gel, Tris-EDTA acetate (TAE) 1×. The gel was then stained 30 min in 0.5 μg/mL ethidium bromide, then washed 3 times for 15 min in water, and visualized with the Azur Biosystem c200 gel doc (Thermofisher, Waltham, Mass., USA).

Search for phage encoding queuosine and archaeosine biosynthesis proteins: The Viruses nr database from NCBI was queried by three iterations of PSI-BLAST³⁷, default set up as previously suggested⁵⁰, using the proteins referenced in Table 2, known to be involved in Queuosine (Q) or Archaeosine (G⁺) biosynthesis, as well as DpdA from Enterobacteria phage 9g, predicted to be involved in the modification of phage DNA, and another DpdA2 from Vibrio phage nt-1, part of a new family identified in this study.

TABLE 2

| | protein | |
|---|---|---|
| accession # | name | Species |
| WP_001139613 | FolE | Escherichia coli |
| WP_000987944 | QueD | Escherichia coli |
| WP_001199973 | QueE | Escherichia coli |
| WP_000817220 | QueC | Escherichia coli |
| WP_000100421 | QueF | Escherichia coli |
| WP_001266503 | QueA | Escherichia coli |
| WP_001294219 | QueG | Escherichia coli |
| WP_013679609 | Gat-QueC | Thermoproteus uzoniensis |
| BAA80469 | QueF-L | Aeropyrum pernix K1 |
| WP_066380731 | ArcS | Halalkalicoccus paucihalophilus |
| WP_011068173 | QueH | Bifidobacterium longum |
| | | NCC2705 |
| WP_005315061 | DUF3820 | Aeromonas salmonicida |
| | (QueD-like) | |
| YP_009032326 | DpdA | Enterobacteria phage 9g |
| YP_008125322 | DpdA2 | Vibrio phage nt-1 |

The PreQ₀ specific transporter YhhQ²⁷ was also added. For each virus identified with at least one of these genes, a reverse analysis was done (phage genome again the protein list) to ensure that no protein was missed during the first analysis. Each identified ortholog was verified by HHpred³⁸ for its annotation.

Identification of the host and their gene content: The Virus-Host DB⁴⁴ was used to gather the host of each phage identified in this study. For phages not referenced in this database, a manual investigation coupling RefSeq⁴² and the literature was performed (data now shown) Each host identified was queried in the Globi database⁴³ (data not shown) The same analysis was done for the double strand DNA (dsDNA) phages, as only these phages were return in our analysis (data not shown). A list of genomes was created on PubSeed⁴⁵ from the hosts identified to create a new spreadsheet.

Mass spectrometry analysis: DNA analysis was performed as previously but with several modifications¹⁶. Purified DNA (20 μg) was hydrolyzed in 10 mM Tris-HCl (pH 7.9) with 1 mM MgCl2 with Benzonase (20 U), DNase I (4 U), calf intestine phosphatase (17 U) and phosphodiesterase (0.2 U) for 16 h at ambient temperature. Following passage through a 10 kDa filter to remove proteins, the filtrate was lyophilized and resuspended to a final concentration of 0.2 μg/μL (based on initial DNA quantity).

Quantification of the modified 2′-deoxynucleosides (dADG, dQ, dPreQ₀, dPreQ₁ and dG⁺) and the four canonical 2′-deoxyribonucleosides (dA, dT, dG, and dC) was achieved by liquid chromatography-coupled triple quadrupole mass spectrometry (LC-MS/MS) and in-line diode array detector (LC-DAD), respectively. Aliquots of hydrolyzed DNA were injected onto a Phenomenex Luna Omega Polar C18 column (2.1×100 mm, 1.6 μm particle size) equilibrated with 98% solvent A (0.1% v/v formic acid in water) and 2% solvent B (0.1% v/v formic acid in acetonitrile) at a flow rate of 0.25 mL/min and eluted with the following solvent gradient: 12% B for 10 min, 1 min ramp to 100% B for 10 min, 1 min ramp to 2% B for 10 min. The HPLC column was coupled to an Agilent 1290 Infinity DAD and an Agilent 6490 triple quadruple mass spectrometer (Agilent, Santa Clara, Calif.). The column was kept at 40° C. and the auto-sampler was cooled at 4° C. The UV wavelength of the DAD was set at 260 nm and the electrospray ionization of the mass spectrometer was performed in positive ion mode with the following source parameters: drying gas temperature 200° C. with a flow of 14 L/min, nebulizer gas pressure 30 psi, sheath gas temperature 400° C. with a flow of 11 L/min, capillary voltage 3,000 V and nozzle voltage 800 V. Compounds were quantified in multiple reaction monitoring (MRM) mode with the following m/z transitions: 310.1→194.1, 310.1→177.1, 310.1→293.1 for dADG, 394.1→163.1, 394.1→146.1, 394.1→121.1 for dQ, 292.1→176.1, 176.1→159.1, 176.1→52.1 for dPreQ₀, 296.1→163.1, 296.1→121.1, 296.1→279.1 for dPreQ₁, and 309.1→193.1, 309.1→176.1, 309.1→159.1 for dG⁺. External calibration curves were used for the quantification of the modified canonical 2′-deoxynucleosides. The calibration curves were constructed from replicate measurements of eight concentrations of each standard. A linear regression with r2>0.995 was obtained in all relevant ranges. The limit of detection (LOD), defined by a signal-to-noise ratio (S/N)≥3, ranged from 0.1 to 1 fmol for the modified 2′-deoxynucleosides. Data acquisition and processing were performed using MassHunter software (Agilent, Santa Clara, Calif.).

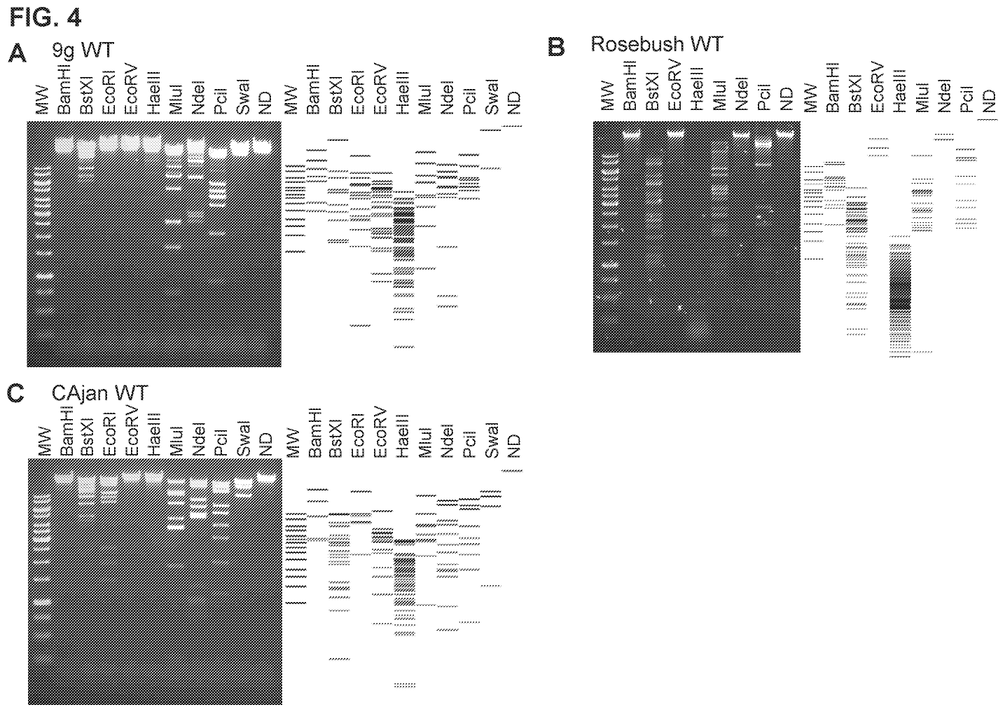

Restriction assay of phage DNA: 250 ng of phage DNA were digested by different enzymes (New England Biolabs) described in FIG. 4 or 1 h at 37° C. in 20 mL CutSmart or 3.1 buffer solution, according to the manufacturer instructions. The enzymes were inactivated by a 20 min incubation at 80° C. The samples were run on a 0.7% agarose gel, Tris-EDTA acetate (TAE) 1×. The gel was then stained 30 min in 0.5 μg/mL ethidium bromide, then wash 3 times for 15 min in water, and visualized with the Azur Biosystem c200 gel doc.

### Example 1—Phage 9g Encodes Functional PreQ₀ Synthesis Genes

First, it was determined whether the phage 9g genes predicted to encode PreQ₀ synthesis enzymes could complement the Q deficiency phenotype of E. coli derivatives lacking the corresponding orthologs. As shown in FIG. 2A, the expression in trans of folE, queD and queE from Enterobacteria phage 9g in E. coli MG1655 ΔfolE, ΔqueD and ΔqueE strains respectively, successfully reestablished the production of queuosine (Q), demonstrating the isofunctionality of the tested pairs. However, this complementation was not observed when the viral gat-queC and dpdA genes were expressed in E. coli ΔqueC and Δtgt, respectively. The result was expected for dpdA as it was predicted to encode an enzyme that recognizes DNA and not tRNA^{14,36}. However, it was unexpected for gGat-QueC, as it was shown previously that expression of an archaeal gat-queC homolog in E. coli could lead to G⁺ in tRNA and hence formation of a PreQ₀ intermediate²⁰.

### Example 2—Phage 9g Gat-QueC and DpdA are Needed for G⁺ Insertion in E. coli DNA Genes

It was predicted that dual expression of the viral gat-queC and dpdA genes in trans would lead to the insertion of 7-deazaguanine derivatives, as dG⁺, in E. coli DNA. Because the presence of dG⁺ confers resistance to EcoRI digestion³⁴, restriction profiles were used as a first indication for the presence of modifications in plasmid DNA. The two phage genes were both cloned in pBAD24 and pBAD33. EcoRI cuts pBAD24 once and pBAD33 twice, as shown in the digestion profiles of plasmids extracted from an E. coli derivative co-transformed with the two empty plasmids (FIG. 2B, lane 1). Because no EcoRI sites are present in the phage 9g gat-queC and dpdA genes, the restriction profiles of plasmids extracted from E. coli derivatives co-transformed with one empty plasmid and one plasmid containing one of the two genes are just shifted by the insert sizes with no additional bands (FIG. 2B, lanes 2, 3, 5 and 6). However, an additional band corresponding to the uncut plasmid was observed for plasmid preparations from strains expressing both gat-queC and dpdA genes (FIG. 2B, lanes 4 and 7). This band only appeared when the genes are induced (FIG. 2C).

Analysis of dG⁺, dADG, dPreQ₀ and dPreQ₁ profiles by liquid chromatography-coupled triple quadrupole mass spectrometry (LC-MS/MS) (FIG. 2B, only dPreQ₀ and dG+ are presented as no dADG or dQ were found) revealed that plasmid DNA extracted from strains expressing only dpdA contained dPreQ₀, plasmid DNA extracted from strains expressing dpdA and gat-queC contained dG⁺ (FIG. 2B, lane 4 and 7), and dPreQ₀ when gat-queC was expressed at lower levels than dpdA (FIG. 2B, lane 4). Taken together, these results showed that dG⁺ but not PreQ₀ could confer resistance to EcoRI and that the phage 9g pathway that inserts dG⁺ in its viral DNA can be transferred to E. coli genomic DNA.

Interestingly, whereas we had failed to complement the Q⁻ phenotype of the E. coli queC strain when expressing the phage 9g Gat-QueC gene, the EcoRI resistance phenotype caused by 7-deazapurine insertion in strains expressing both 9g dpdA and gat-queC was still observed in a ΔqueC background (FIG. 2B, lanes 8 and 9) but not in a ΔqueD background (FIG. 2B, lanes 10, 11). Furthermore, only dG⁺ modification was observed in DNA of the ΔqueC strains by LC-MS/MS. This suggests that the Gat-QueC protein can produce PreQ₀ but that it is channeled to the putative DNA modifying enzyme DpdA and not to the tRNA modifying pathway enzyme QueF.

Finally, whether the E. coli TGT was required for DpdA activity in E. coli was tested as the active forms of TGT enzymes are known to be dimers³⁶. This does not seem to be the case as the restriction resistance phenotype was still observed in the Δtgt background (FIG. 2B, lanes 12 and 13).

### Example 3—a Wide Variety of Phages Harbor the dG⁺ Biosynthesis Pathway

A new sub-family of DpdA encoded by the Vibrio phage nt-1 was identified by investigating genes flanking PreQ₀ biosynthesis genes cluster. Indeed, phage nt-1 DpdA (YP_008125322) is not detected with PSI-BLAST when using the E. coli phage 9g DpdA as input sequence and it does not possess the conserved histidine found at position 196 but similarities with members of the TGT family could be detected using HHpred. This protein was renamed DpdA2.

An in silico search for phages that could harbor 7-deazaguanine derivatives in their genomic DNA revealed that a total of 182 viruses deposited in GenBank were found to encode a DpdA homolog and/or at least a G⁺ synthesis gene (Table 1). Most of these viruses (163/182) were bacteriophages, while 16 archaeal viruses as well as the 3 eukaryotic viruses were found. The latter only encode for FolE, which is most likely to be linked to the folate pathway³⁹. Analyses of the presence/absence patterns of the predicted Q/G⁺ biosynthesis genes led to classification of these viruses in various groups and in some cases, predict the nature of the 7-deazaguanine base modification. It is important to note that no homologs to the proteins specifically involved in Q biosynthesis such as QueA, QueG, or QueH (see FIG. 1) were found in viruses.

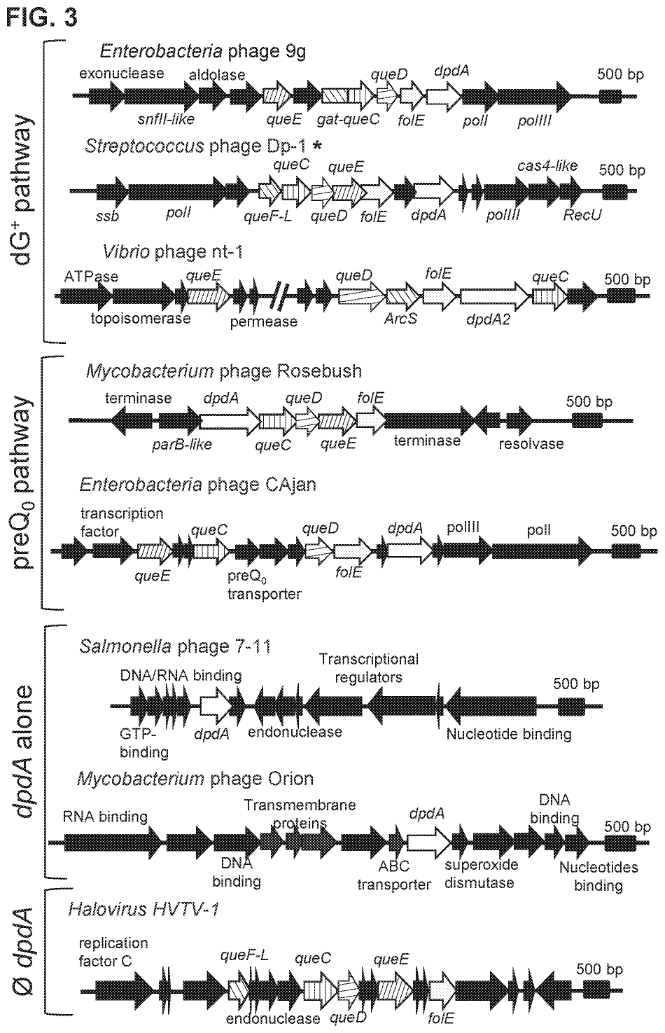

The first group contains 25 phages and is represented by Enterobacteria phage 9g (KJ419279), Streptococcus phage Dp-1 (NC_015274) and Vibrio phage nt-1 (NC_021529) in FIG. 3. Those phages encode homologs of 9g DpdA or nt-1 DpdA2 as well as homologs of FolE, QueD, QueE and QueC. In addition, they encode homologs of one of the three amidotransferases involved in the last steps of G⁺ synthesis: ArcS, QueF-L (or QueF) or a Gat-QueC fusion, which replace the canonical QueC in this last case. These phages likely modify their DNA with dG⁺, as phage 9g¹⁴ does. It should be noted that the discrimination between the QueF-L homologs, predicted to produce the G⁺ base from PreQ₀, and QueF homologs, predicted to produce PreQ₁ from PreQ₀, is difficult to establish based on the sequence similarity only. Therefore, the genome of phages encoding for these proteins might harbor dG⁺ or dPreQ₁ (or both).

The second group includes 40 phages and is represented by E. coli phage CAjan (NC_028776) and Mycobacterium phage Rosebush (AY129334) in FIG. 3. These phages encode a homolog of one of the two types of DpdA, and of the PreQ₀ synthesis enzymes (FolE, QueD, QueE and QueC), but they are missing an amidotransferase. As such, it is predicted that these phages modify their DNA with PreQ₀ or ADG, like the bacteria that contain the dpd cluster¹⁴. Mycobacterium phage Bipper (KU728633) that misses only a gene encoding QueC was added to this group even if it could be modified by the QueC substrate (CDG, see FIG. 1). The uncultured phage clone 7AX_2 (MF417872) was also added to this group as it also lacks a gene encoding QueC, although this may be due to the incomplete genomic sequence of this phage. Whether this phage also encodes an amidotransferase could not be excluded.

The third group contains 76 phages including Salmonella phage 7-11 (NC_015938) and Mycobacterium phage Orion (DQ398046) shown in FIG. 3. These phages encode DpdA but no G⁺ or PreQ₀ biosynthesis protein homologs. At this stage, their genome modification status, if any, was difficult to predict. Phages in this group could rely on PreQ₀ synthesized by the host or on uptake of exogenous 7-deazaguanine precursors. The large size of this group compared to the others might be caused by the relatively large number of Mycobacteriophages in the virus database due to the massive phage isolation and sequencing effort of PhagesDB and the SEA-PHAGES project.

The last group is composed of 48 phages encoding proteins of the PreQ₀/G⁺ pathway but no DpdA. These phages could boost the production of the Q precursor to increase the level of Q in the host tRNA and increase translation efficiency⁴⁰. However, it is possible that 7-deazaguanines are inserted in their DNA in a DpdA independent pathway as there is a recent report that the genomes of Capylobacter phages from this group are highly modified by dADG (data not shown).

Phages containing FolE and QueC singletons were discarded from further analysis because FolE is shared between folate and PreQ₀ synthesis¹⁶ while QueC is also part of a superfamily of ATPase (COG) making their precise role to identify.

All the phages identified above are members of the Caudovirales order and are distributed into various families: Siphoviridae (95), Myoviridae (23), Ackermannviridae (20) and Podoviridae (3). For the Archaeal virus, 12 Ligamenvirales and 2 Bicaudaviridae were identified (data not shown).

### Example 4—the Host May Participate in the Phage DNA Modification

To study the interaction between phages containing 7-deazaguanine related genes and their bacterial hosts, metadata on the hosts and their habitat was gathered using RefSeq⁴² and the Globi database⁴³, and the distribution of Q, G⁺ and dADG synthesis genes in these organisms was analyzed (data not shown). Interestingly, 106 of the collected phages (˜60%) infect a strain that is the model for a known bacterial pathogen, where only ˜9% of the dsDNA viruses from the Virus-Host database⁴⁴ infect a strain related to pathogen (data not shown). No clear environment was found for the archaeal hosts.

All phage hosts predicted to modify their DNA with G⁺ possess the pathway to produce Q in tRNA. Curiously the hosts of the phages coding for a QueF-L and a 9g DpdA homolog do not encode for the PreQ₀ biosynthetic pathway (QueDEC, see FIG. 1), but encode for the specific PreQ₀ transporter YhhQ and the rest of the Q pathway (QueFAG and TGT, FIG. 1). Conversely, all the hosts of the DpdA2 encoding phages encode the full Q pathway. As shown in FIG. 1, 7-cyano-7-deazaguanine (PreQ0) is synthesized from GTP by four enzymes (FolE, QueD, QueE, QueC) and is the key intermediate in both the Q and G⁺ pathways. The last step of PreQ₀ synthesis is catalyzed by 7-cyano-7-deazaguanine synthase (QueC) in a complex reaction that goes through the 7-amido-7-deazaguanine (ADG) intermediate. tRNA-guanine-transglycosylases (TGT in bacteria, arcTGT in archaea) are the signature enzymes in the Q and G+ tRNA modification pathways as they exchange the targeted guanines with the 7-deazaguanine precursors. In archaea, PreQ₀ is directly incorporated into tRNA by arcTGT before being further modified by different types of amidotransferases (ArcS, Gat-QueC or QueF-L). In bacteria, PreQ0 is reduced to 7-aminomethyl-7-deazaguanine (PreQ₁) by QueF before TGT incorporates it in tRNA, where it is further modified to Q in two steps (FIG. 1).

There is no clear pattern for the bacterial hosts of phages encoding both DpdA and the whole PreQ₀ pathway. Most of them encode the full Q pathway enzymes except for Streptococcus pneumoniae, which lacks PreQ₀ pathway genes, Rhodococcus erythropolis, which encodes only TGT, and the Mycobacteria, that possess none of these genes.

The hosts of the phages encoding only DpdA also encode for the full set of Q synthesis enzymes except the Clostridium species, which lack the PreQ₀ pathway genes, and the Mycobacterium genus, that possess none of these genes. Sulfolobi were not referenced in PubSeed⁴⁵, but using BLASTp with default parameters with the genes listed in Table 2 above as queries, all G⁺ pathway genes were identified. Hence, the 7-deazaguanine intermediates produced by these hosts, Clostridium and Mycobacterium excluded, might be used by phages that lack the biosynthesis proteins to produce a 7-deazaguanine precursor.

Finally, the hosts of the phages that do not encode a DpdA but encode the PreQ₀ pathway proteins all encode the full Q synthesis pathway.

A few bacterial hosts, such as 46 different strains of E. coli, Haloarcula valismortis and Vibrio harveyi 1DA3, also harbor homologs of the bacterial DpdA. In these cases, infecting phages could be modified by the host modification machinery.

### Example 5—Different Set of Genes for Different 7-Deazaguanine Modifications

To test predictions on the nature of phage DNA modification, a set of phages from each group was selected, and their genomic DNA were extracted for mass spectrometry analysis (Table 3).

TABLE 3

| | | | Prediction | | | | | |
|---|---|---|---|---|---|---|---|---|
| Phage/Virus | | | based on | | | | | |
| | | GC | gene | Modification per 10⁶ nuclotides | | | | |
| Accession # | Name | content | content | dpreQ₀ | dADG | dG⁺ | dpreQ₁ | dQ |
| NC_028776 | Escherichia | 44.7% | dPreQ₀ | 70628 | None | None | None | None |
| | phage CAjan | | | | | | | |
| None | Escherichia | | None | None | None | None | None | None |
| | phage CAjan | | | | | | | |
| | ΔdpdA | | | | | | | |
| NC_020158 | Halovirus | 58.3% | None/dG⁺ | None | 152 | 22 | 420654 | None |
| | HVTV-1 | | | | | | | |
| NC_008197 | Mycobacterium | 66.5% | None | None | None | None | None | None |
| | phage Orion | | | | | | | |
| NC_004684 | Mycobacterium | 69.0% | dPreQ₀ | 96530 | 9 | None | None | None |
| | phage | | | | | | | |
| | Rosebush | | | | | | | |
| NC_015938 | Salmonella | 44.1% | None/PreQ₀ | None | 50 | None | None | None |
| | phage 7-11 | | | | | | | |
| NC_015274 | Streptococcus | 40.3% | dPreQ₁/dG⁺ | None | None | None | 9605 | None |
| | phage Dp-1 | | | | | | | |
| NC_021529 | Vibrio phage | 41.3% | dG⁺ | 232 | 72 | 44 | None | None |
| | nt-1 | | | | | | | |

Interestingly, no 2′-deoxyqueuosine (dQ) was found in any of the tested samples, correlating with the fact that no phage or virus encodes the specific protein for Q synthesis (QueAGH).

First, phages encoding both a DpdA and one of the amidotransferase homologs were analyzed. Streptococcus phage Dp-1 DNA, encoding for a QueF-L, contained a large amount of dPreQ₁ (3,389 modifications per 10⁶ nucleotides, ˜1.7% of the Gs) but no dG⁺, which would mean that the QueF-L of this phage would actually be functionally closer to the bacterial QueF than the archaeal QueF-L, as predicted by the SSN clustering. Vibrio phage nt-1, encoding an ArcS, was shown to harbor not only dG⁺ (44 modifications per 10⁶ nucleotides, ˜0.02% of the Gs) but also dPreQ₀ and dADG (232 modifications per 10⁶ nucleotides, ˜0.11% of the Gs, and 72 modifications per 10⁶ nucleotides, ˜0.03% of the Gs, respectively). This result might indicate that nt-1 DpdA is more promiscuous and could insert all intermediates of the pathway.

Next, phages of the second group that encode both a DpdA and the four proteins of the PreQ₀ biosynthesis pathway but no amidotransferase homolog were investigated. Mycobacterium phage Rosebush was found to harbor dPreQ₀ in its DNA (96,530 modifications per 10⁶ nucleotides, ˜28% of the Gs) as does Escherichia phage CAjan (70,628 modifications per 10⁶ nucleotides, ˜32% of the Gs). However, Mycobacterium phage Rosebush was found to also harbor a very small amount of dADG (9 modifications per 10⁶ nucleotides, ˜0.003% of the Gs). These proportions are negligible for Rosebush and could be the result of the natural oxidation of the PreQ₀ base.

The genomic DNA of Salmonella phage 7-11 and Mycobacterium phage Orion from the third group of phage, which only encode a DpdA were also analyzed by LC-MS/MS. Mycobacterium phage Orion lacked any 7-deazaguanine modifications in its DNA. This result was expected as none of the phage nor the host encode for the PreQ₀ biosynthesis pathway (Mycobacterium smegmatis, Table 3). However, Salmonella phage 7-11 was unexpectedly modified by dADG (50 modifications per 10⁶ nucleotides, ˜0.02% of the Gs), suggesting the presence of a protein responsible for the oxidation of PreQ₀ encoded by the phage.

Finally, Halovirus HVTV-1, which encodes the four proteins of the PreQ₀ biosynthesis pathway and an ArcS homolog but no DpdA, contained mainly dPreQ₁ (88,607 modifications per 10⁶ nucleotides, ˜30% of the Gs) but also relatively small amounts of dADG and dG⁺ (152 modifications per 10⁶ nucleotides, ˜0.05% of the Gs, and 22 modifications per 10⁶ nucleotides, ˜0.008% of the Gs, respectively). As its host, Haloarcula valismortis, harbors a DpdA homolog, it is possible that the host DpdA inserts PreQ₀ in Halovirus HVTV-1 DNA before it is further modified to dPreQ₁ or dG⁺ by the viral ArcS, that would have evolved to perform a nitrile reduction as well, or to dADG by another unidentified protein.

### Example 6—Exemplary Modifications Protect the Phage Genome from the Restriction

The different modifications present in the phages analyzed above may lead to distinct resistance patterns to host defense mechanism such as RM systems. To test this hypothesis, phage DNA preparations were digested with a set of restriction enzymes that had been shown to be totally or partially inactivated in the presence of the dG⁺ modification³⁴. As a control, and as shown in FIG. 4A, no digestion was observed with BamHI, EcoRI, EcoRV, and SwaI while it was partially restricted with BstXI, HaeIII, MluI, NdeI, PciI.

Mycobacteria phage Rosebush DNA that carries PreQ₀ showed a slightly different pattern of resistance. The restriction profiles for BamHI, BstXI and EcoRV were identical to those of Enterobacteria phage 9g. However, Rosebush DNA was fully sensitive to HaeIII, MluI and PciI and resisted to NdeI degradation (FIG. 4B). EcoRI and SwaI could not be tested as the corresponding sites are absent in the Mycobacterium phage Rosebush genome.

Discussion:

As described herein, the presence of 7-deazaguanine modifications was directly linked with a restriction resistance phenotype.

In addition, all 7-deazaguanine modified DNA preparation tested were protected to various degrees from digestion by restriction enzymes. Transplanting the dG⁺ modification in E. coli reproduced the resistance to cleavage by EcoRI (FIG. 2).

Four 7-deazaguanine modifications in DNA were detected: dADG in bacteria, and dG⁺, dPreQ₁ and dPreQ₀, all represented in phages. dADG was observed in phage genomes for the first time. The genes involved in the synthesis of these different modifications also were identified. FolE, QueD and QueE from Enterobacteria phage 9g were proven to functionally replace their E. coli orthologs (FIG. 2A).

Most 7-deazaguanine containing phage genomes also harbor a gene coding for a DpdA homolog. As with its bacterial homolog³², the phage DpdA introduces PreQ₀ in DNA (FIG. 2B), most probably through a base exchange mechanisms similar to its TGT homolog³⁶. DpdA2 proteins appear to share this function, as Vibrio phage nt-1 genome contains dPreQ₀. However, not all phages/viruses containing 7-deazaguanines encodes DpdA proteins, as seen with Halovirus HVTV-1 (Table 3 above). It is possible that in the HVTV-1 case, the host DpdA is responsible for the presence of modifications in its genome (EMA11768 in AOLQ01000002). Still, a DpdA is not always present in the host, and there could be cases where the phages encode a machinery to create modified dGTP for the DNA polymerase to use, as proposed for Campylobacter phages (data not shown). Finally, one cannot rule out that some phages may harbor new families of 2′-deoxyribosyltransferase to be discovered.

The combination of comparative genomic analyses and experimental validations described herein has allowed to elucidate pathways for the insertion of dPreQ₀, dPreQ₁ and dG⁺ in phage genomes (FIG. 5). The presence of the minimal set of FolE, QueD, QueE QueC and DpdA proteins leads to the insertion of dPreQ₀, as seen in Mycobacterium phage Rosebush (Table 3 above). The replacement of QueC by Gat-QueC leads to the introduction of dG⁺ (FIG. 2B). However it is not known if Gat-QueC converts PreQ₀ into G⁺ before or after it is inserted in DNA. The function of ArcS homologs in phages/viruses is less clear. Indeed, Vibrio phage nt-1 encodes an ArcS homolog and its DNA contains mostly. dPreQ₀ but also dG⁺ and dADG (FIG. 5). ArcS was the first G⁺ synthase identified in archaea¹⁹. It is possible that some phage ArcS protein evolved to perform not only an amidotransferase reaction, like the archaeal ArcS¹⁹, but either an nitrile reduction, like the bacterial QueF²², or an amidohydrolase reaction, like the bacterial DpdC³².

HHpred analysis predicted that a homolog of the archaeal QueF-L, that synthesizes G⁺-tRNA from the PreQ₀-tRNA⁴⁹, was encoded by Streptococcus phage Dp-1. However, we found that this phage was modified by dPreQ₁. It is unclear if the reduction occurs on free PreQ₀, similarly to the bacterial QueF proteins²², and then the free base PreQ₁ is inserted by DpdA, or if the phage QueF is able to modify the DNA-bounded dPreQ₀, as does the archaeal QueF-L with tRNA⁴⁹. However, Halovirus HVTV-1 contains mainly dPreQ₁, but also small amounts of dADG and dG⁺. It is possible that the QueF-L is on the verge of evolving from an amidohydrolase to an amidotransferase reaction, but one cannot rule out that the host ArcS could catalyze the reaction, although the specific PUA domain specific for tRNA bidding makes it highly unlikely.

Interestingly, 7-deazaguanine modifications seem to dramatically decrease the susceptibility of the phage genomes to the host restriction-modification systems (RM). These systems are one of the major defense systems for bacteria to prevent the invasion by foreign DNA⁵. Phages evolved to escape these RM systems by different methods including modification of their genomic DNA¹¹⁻¹⁴. As demonstrated by the data provided herein, the presence of the dG⁺ modification was directly linked with the restriction resistance phenotype. In addition, all 7-deazaguanine modified DNA preparations tested were protected to various degrees from digestion by restriction enzymes. It was also observed that introducing the dG⁺ modification in E. coli reproduced the resistance to cleavage by EcoRI (FIG. 2).

### Example 7—In Vivo Modification System

The following Example describes an in vivo method for introducing 7-deazaguanine modifications into a heterologous nucleic acid.

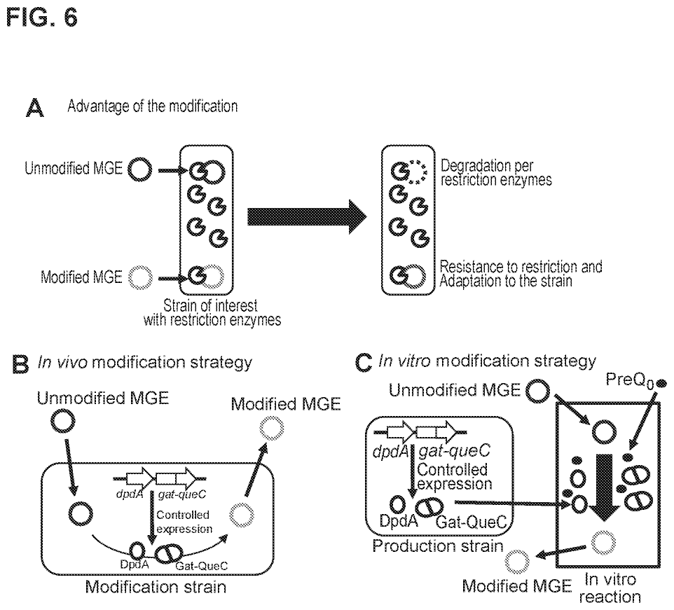

Specific laboratory strains of the gram-negative bacteria Escherichia coli and the gram positive Bacillus subtilis will be engineered to encode the dpdA and gat-queC from Enterobacteria phage 9g and produce the respective proteins, DpdA and Gat-QueC, when voluntarily induced by the experimenter (FIG. 6B). The MGE of interest can be then inserted in this strain, by transformation or conjugation for plasmids and integrons, or regular infection for phages, to be modified by dG⁺, as seen in FIG. 2. The MGE can then be collected by lysing the cells and will be ready to used to be introduced in the strain of interest. A system encoding only dpdA will also be created to obtain the dPreQ₀, and the necessary genes to produce dPreQ1 will be investigated to create a system inducing this modification.

The advantage of this system is that it requires only a few materials but the strain of interest has to have a compatible MGE with the modifying strain. The number of modifying strains used to produce the modification will be expanded as this technology grows to be more available to diverse species of bacteria.

### Example 8—In Vitro Modification System

The following Example describes an in vitro method for introducing 7-deazaguanine modifications into a heterologous nucleic acid.

The Enterobacteria phage 9g dpdA and gat-queC genes will be cloned in an expression plasmid, such as pET28. DpdA and Gat-QueC protein will be expressed in a specific strain of E. coli, such as BL21, and further purified to be used in vitro (FIG. 6C). The MGE DNA will be mixed with the two purified enzymes and with the PreQ0 base and incubated to promote the modification of the MGE DNA by dG+, as seen in vivo in FIG. 2. The MGE can be purified and introduced into the strain of interest. The use of DpdA alone will provide a MGE modified with dPreQ0, and the protein necessary for dPreQ1 will be purified to obtain this modification.

The advantage of this method is that all that is needed is the proteins and PreQ0 to modify a nucleic acid of interest, and thus it can be easily set up in form of a kit. However, this technique is not applicable to phage, unless the phage packaging system is available in vitro.

### REFERENCES CITED IN THE EXAMPLES

1. Chopin, M. C., Chopin, A. & Bidnenko, E. Phage abortive infection in lactococci: Variations on a theme. Curr. Opin. Microbiol. 8, 473-479 (2005).
2. Labrie, S. J., Samson, J. E. & Moineau, S. Bacteriophage resistance mechanisms. Nat. Rev. Microbiol. 8, 317-327 (2010).
3. Golais, F., Hollý, J. & Vitkovská, J. Coevolution of bacteria and their viruses. Folia Microbiol. (Praha). 58, 177-186 (2013).
5. Ershova, A. S., Rusinov, I. S., Spirin, S. A., Karyagina, A. S. & Alexeevski, A. V. Role of restriction-modification systems in prokaryotic evolution and ecology. Biochem. 80, 1373-1386 (2015).
6. Chaudhary, K. BacteRiophage EXclusion (BREX): A novel anti-phage mechanism in the arsenal of bacterial defense system. J. Cell. Physiol. 233, 771-773 (2018).
7. Doron, S. et al. Systematic discovery of antiphage defense systems in the microbial pangenome. Science (80-.). 359, 0-12 (2018).
8. Samson, J. E., Magadan, A. H., Sabri, M. & Moineau, S. Revenge of the phages: Defeating bacterial defences. Nat. Rev. Microbiol. 11, 675-687 (2013).
9. Borges, A. L., Davidson, A. R. & Bondy-Denomy, J. The Discovery, Mechanisms, and Evolutionary Impact of Anti-CRISPRs. Annu. Rev. Virol. 4, annurev-virology-101416-041616 (2017).
10. Pawluk, A., Davidson, A. R. & Maxwell, K. L. Anti-CRISPR: Discovery, mechanism and function. Nat. Rev. Microbiol. 16, 12-17 (2018).
11. Bryson, A. L. et al. Covalent Modification of Bacteriophage T4 DNA Inhibits CRISPR-Cas9. MBio 6, e00648-15 (2015).
12. Weigele, P. & Raleigh, E. A. Biosynthesis and Function of Modified Bases in Bacteria and Their Viruses. (2016). doi:10.1021/acs.chemrev.6b00114
13. Lee, Y.-J. et al. Identification and biosynthesis of thymidine hypermodifications in the genomic DNA of widespread bacterial viruses. Proc. Natl. Acad. Sci. 201714812 (2018). doi:10.1073/pnas.1714812115
14. Thiaville, J. J. et al. Novel genomic island modifies DNA with 7-deazaguanine derivatives. Proc. Natl. Acad. Sci. U.S.A 113, E1452-9 (2016).
15. Reader, J. S., Metzgar, D., Schimmel, P. & De Crecy-Lagard, V. Identification of Four Genes Necessary for Biosynthesis of the Modified Nucleoside Queuosine. J. Biol. Chem. 279, 6280-6285 (2004).
16. Phillips, G. et al. Biosynthesis of 7-deazaguanosine-modified tRNA nucleosides: A new role for GTP cyclohydrolase I. J. Bacteriol. 190, 7876-7884 (2008).
17. McCarty, R. M. & Bandarian, V. Biosynthesis of pyrrolopyrimidines. Bioorg. Chem. 43, 15-25 (2012).
18. Nelp, M. T. & Bandarian, V. A Single Enzyme Transforms a Carboxylic Acid into a Nitrile through an Amide Intermediate. Angew. Chemie Int. Ed. n/a-n/a (2015). doi:10.1002/anie.201504505
19. Phillips, G. et al. Discovery and characterization of an amidinotransferase involved in the modification of archaeal tRNA. J. Biol. Chem. 285, 12706-12713 (2010).
20. Phillips, G. et al. Diversity of archaeosine synthesis in crenarchaeota. ACS Chem. Biol. 7, 300-305 (2012).
21. Bon Ramos, A., Bao, L., Turner, B., de Crecy-Lagard, V. & Iwata-Reuyl, D. QueF-Like, a Non-Homologous Archaeosine Synthase from the Crenarchaeota. Biomolecules 7, 1-14 (2017).
22. Van Lanen, S. G. et al. From cyclohydrolase to oxidoreductase: Discovery of nitrile reductase activity in a common fold. Proc. Natl. Acad. Sci. U.S.A 102, 4264-4269 (2005).
23. Stengl, B., Reuter, K. & Klebe, G. Mechanism and substrate specificity of tRNA-guanine transglycosylases (TGTs): tRNA-modifying enzymes from the three different kingdoms of life share a common catalytic mechanism. ChemBioChem 6, 1926-1939 (2005).
24. Van Lanen, S. G. & Iwata-Reuyl, D. Kinetic mechanism of the tRNA-modifying enzyme S-adenosylmethionine:tRNA ribosyltransferase-isomerase (QueA). Biochemistry 42, 5312-5320 (2003).
25. Miles, Z. D., McCarty, R. M., Molnar, G. & Bandarian, V. Discovery of epoxyqueuosine (oQ) reductase reveals parallels between halorespiration and tRNA modification. Proc. Natl. Acad. Sci. U.S.A 108, 7368-72 (2011).
26. Zallot, R. et al. Identification of a Novel Epoxyqueuosine Reductase Family by Comparative Genomics. ACS Chem. Biol. 12, 844-851 (2017).
27. Zallot, R., Yuan, Y. & De Crecy-Lagard, V. The Escherichia coli COG1738 member YhhQ is involved in 7-cyanodeazaguanine (preQ0) transport. Biomolecules 7, 1-13 (2017).
28. Carstens, A. B., Kot, W. & Hansen, L. H. Complete Genome Sequences of Four Novel Escherichia coli Bacteriophages Belonging to New Phage Groups. Genome Announc. 3, e00741-15 (2015).
29. Sabri, M. et al. Genome annotation and intraviral interactome for the Streptococcus pneumoniae virulent phage Dp-1. J. Bacteriol. 193, 551-562 (2011).
30. Kot, W. et al. Complete Genome Sequence of Streptococcus pneumoniae Virulent Phage MS1. Genome Announc. 5, 9-10 (2017).
31. Pedulla, M. L. et al. Origins of highly mosaic mycobacteriophage genomes. Cell 113, 171-182 (2003).
32. Yuan, Y. et al. Identification of the minimal bacterial 2′-deoxy-7-amido-7-deazaguanine synthesis machinery. Molecular Microbiology (2018). doi:10.1111/mmi.14113
33. Kulikov, E. et al. Genomic Sequencing and Biological Characteristics of a Novel Escherichia Coli Bacteriophage 9g, a Putative Representative of a New Siphoviridae Genus. Viruses 6, 5077-5092 (2014).
34. Tsai, R., Correa, I. R., Xu, M. Y. & Xu, S. Y. Restriction and modification of deoxyarchaeosine (dG+)-containing phage 9 g DNA. Sci. Rep. 7, 1-13 (2017).
35. Mačková, M., Boháčová, S., Perlíková, P., Poštová Slavětinská, L. & Hocek, M. Polymerase Synthesis and Restriction Enzyme Cleavage of DNA Containing 7-Substituted 7-Deazaguanine Nucleobases. ChemBioChem 16, 2225-2236 (2015).
36. Hutinet, G., Swarjo, M. A. & de Crécy-Lagard, V. Deazaguanine derivatives, examples of crosstalk between RNA and DNA modification pathways. RNA Biol. 14, 1175-1184 (2017).
37. Altschul, S. F. et al. Gapped BLAST and PSI-BLAST: A new generation of protein database search programs. Nucleic Acids Res. 25, 3389-3402 (1997).
38. Söding, J. Protein homology detection by HMM-HMM comparison. Bioinformatics 21, 951-960 (2005).
39. Hanson, A. D. & Gregory, J. F. Synthesis and turnover of folates in plants. Curr. Opin. Plant Biol. 5, 244-249 (2002).
40. Tuorto, F. et al. Queuosine-modified tRNAs confer nutritional control of protein translation. EMBO J. e99777 (2018). doi:10.15252/embj.201899777
41. Cicmil, N. & Huang, R. H. Crystal structure of QueC from Bacillus subtilis: An enzyme involved in preQ1 biosynthesis. Proteins Struct. Funct. Genet. 72, 1084-1088 (2008).
42. O'Leary, N. A. et al. Reference sequence (RefSeq) database at NCBI: Current status, taxonomic expansion, and functional annotation. Nucleic Acids Res. 44, D733-D745 (2016).
43. Poelen, J. H., Simons, J. D. & Mungall, C. J. Global biotic interactions: An open infrastructure to share and analyze species-interaction datasets. Ecol. Inform. 24, 148-159 (2014).
44. Mihara, T. et al. Linking virus genomes with host taxonomy. Viruses 8, 10-15 (2016).
45. Overbeek, R. et al. The subsystems approach to genome annotation and its use in the project to annotate 1000 genomes. Nucleic Acids Res. 33, 5691-5702 (2005).
46. Carstens, A. B., Kot, W., Lametsch, R., Neve, H. & Hansen, L. H. Characterisation of a novel enterobacteria phage, CAjan, isolated from rat faeces. Arch. Virol. 161, 2219-2226 (2016).
47. Lemay, M.-L., Renaud, A., Rousseau, G. & Moineau, S. Targeted Genome Editing of Virulent Phages Using CRISPR-Cas9. Bio-Protocol 7, 1-19 (2018).
48. Loenen, W. A. M. Tracking EcoKI and DNA fifty years on: A golden story full of surprises. Nucleic Acids Res. 31, 7059-7069 (2003).
49. Mei, X. et al. Crystal Structure of the Archaeosine Synthase QueF-Like-Insights into Amidino Transfer and tRNA Recognition by the Tunnel Fold. Proteins 165, 255-269 (2016).
50. Lopes, A., Amarir-Bouhram, J., Faure, G., Petit, M. A. & Guerois, R. Detection of novel recombinases in bacteriophage genomes unveils Rad52, Rad51 and Gp2.5 remote homologs. Nucleic Acids Res. 38, 3952-3962 (2010).
51. Altenhoff, A. M. et al. The OMA orthology database in 2018: Retrieving evolutionary relationships among all domains of life through richer web and programmatic interfaces. Nucleic Acids Res. 46, D477-D485 (2018).
52. Gerlt, J. A. et al. Enzyme function initiative-enzyme similarity tool (EFI-EST): A web tool for generating protein sequence similarity networks. Biochim. Biophys. Acta—Proteins Proteomics 1854, 1019-1037 (2015).
53. Shannon, P. et al. Cytoscape: a software environment for integrated models of biomolecular interaction networks. Genome Res. 2498-2504 (2003). doi:10.1101/gr.1239303.metabolite
54. Levic, J. & Micura, R. Syntheses of 15N-Labeled pre-queuosine nucleobase derivatives. Beilstein J. Org. Chem. 10, 1914-1918 (2014).
55. Lemay, M. L., Tremblay, D. M. & Moineau, S. Genome Engineering of Virulent Lactococcal Phages Using CRISPR-Cas9. ACS Synth. Biol. 6, 1351-1358 (2017).
56. Kot, W., Vogensen, F. K., Sorensen, S. J. & Hansen, L. H. DPS—A rapid method for genome sequencing of DNA-containing bacteriophages directly from a single plaque. J. Virol. Methods 196, 152-156 (2014).

What is claimed is:

1. A bacterial cell comprising a heterologous nucleic acid sequence comprising one or more deazapurine bases.

2. The bacterial cell of claim 1, wherein the one or more deazapurine bases are deazaguanine bases.

3. The bacterial cell of claim 1, wherein the deazaguanine bases are 7-deazaguanine bases

4. The bacterial cell of claim 3, wherein the one or more 7-deazaguanine bases are 7-amido-7-deazaguanine (ADG), 7-formamidino-7-deazaguanosine (G⁺), 7-cyano-7-deazaguanine (PreQ0) and/or 7-aminomethyl-7-deazaguanine (PreQ1).

5. The bacterial cell of claim 4, wherein the deazaguanine bases are 7-formamidino-7-deazaguanosine (G⁺) or 7-cyano-7-deazaguanine (PreQ₀).

6. The bacterial cell of claim 1, wherein the bacterial cell is an E. coli bacterial cell or a B. cereus bacterial cell.

7. The bacterial cell of any one of claims 1-6, wherein the heterologous nucleic acid sequence is incorporated into the bacterial genome.

8. A method of protecting a heterologous nucleic acid sequence from cleavage by restriction enzymes in a host bacterium, the method comprising:
modifying the heterologous nucleic acid sequence to incorporate one or more deazaguanine bases; and
introducing the modified heterologous nucleic acid sequence into the host bacterium, thereby protecting the heterologous nucleic acid sequence from cleavage by restriction enzymes in the host bacterium.

9. The method of claim 8, wherein the modifying step comprises mixing the heterologous nucleic acid sequence with a transglycosidase, an amidotransferase and 7-cyano-7-deazaguanine (PreQ₀) for a time sufficient to promote modification of the heterologous nucleic acid sequence.

10. The method of claim 9, wherein the amidotransferase is Gat-QueC.

11. The method of claim 9, wherein the transglycosidase is DpdA.

12. The method of claim 8, wherein the modifying step comprises introducing the heterologous nucleic acid into a bacterial cell that has been modified to encode a transglycosidase and an amidotransferase.

13. The method of any one of claims 8-12, wherein the deazaguanine bases are 7-deazaguanine bases.

14. The method of claim 13 wherein the one or more 7-deazaguanine bases are 7-amido-7-deazaguanine (ADG), 7-formamidino-7-deazaguanosine (G⁺), 7-cyano-7-deazaguanine (PreQ₀) and/or 7-aminomethyl-7-deazaguanine (PreQ₁).

15. A method of producing a bacteriophage composition, the method comprising (a) modifying a nucleic acid of bacteriophage origin to incorporate one or more deazaguanine bases; (b) introducing the modified nucleic acid into a host bacteria cell; (c) incubating the host bacteria cell until phage-mediated bacterial lysis occurs; and (d) isolating bacteriophage lysate.


SEQUENCE LISTING (1 sequence)

SEQ ID NO: 1 - DNA, 18 bp, Artificial Sequence
  Synthetic Polypeptide
  misc_feature (1)..(1): Biotin
ccctcggtgacaggcagg